(19) United States
(12) Patent Application Publication
(10) Pub. No.: US 2014/0094486 A1
(43) Pub. Date: Apr. 3, 2014

(54) SOLID STATE FORMS OF HIV INHIBITOR

(71) Applicant: Gilead Sciences, Inc., Foster City, CA (US)

(72) Inventors: Zheng Jane LI, Southbury, CT (US); Zhibin LI, Berlin, CT (US); Laibin LUO, Pierrefonds (CA); Thomas OFFERDAHL, Torrington, CT (US); Soojin KIM, Demarest, NJ (US); Bing-Shiou YANG, Southbury, CT (US)

(21) Appl. No.: 14/045,037

(22) Filed: Oct. 3, 2013

Related U.S. Application Data
(63) Continuation of application No. PCT/US2012/032026, filed on Apr. 3, 2012.
(60) Provisional application No. 61/481,908, filed on May 3, 2011.
(60) Provisional application No. 61/471,655, filed on Apr. 4, 2011.

(51) Int. Cl. A61K 31/436; A61K 45/06; C07D 491/06

(52) U.S. Cl. CPC A61K 31/436; C07D 491/06; A61K 45/06
USPC 514/291; 546/89

(57) ABSTRACT

The invention relates to novel crystalline forms of (2S)-2-tert-butoxy-2-(4-(2,3-dihydropyrano[4,3,2-de]quinolin-7-yl)-2-methylquinolin-3-yl)acetic acid, the hydrochloride salt thereof, novel crystalline forms of the hydrochloride salt, methods for the preparation thereof, pharmaceutical compositions thereof and their use in the treatment of Human Immunodeficiency Virus (HIV) infection.

### CROSS-REFERENCE TO RELATED APPLICATIONS

This application is a continuation of International PCT Patent Application No. PCT/2012/032026, which was filed on Apr. 3, 2012, now pending, which claims the benefit under 35 U.S.C. §119(e) of U.S. Provisional Patent Application No. 61/471,655, filed Apr. 4, 2011, and U.S. Provisional Patent Application No. 61/481,908, filed May 3, 2011, which applications are incorporated herein by reference in their entireties.

### BACKGROUND

1. Field

This invention relates to a novel salt form of Compound (I), novel crystalline forms of Compound (I) and novel crystalline forms of the hydrochloride salt of Compound (I) as described herein, methods for the preparation thereof, pharmaceutical compositions thereof, and their use in the treatment of Human Immunodeficiency Virus (HIV) infection.

2. Description of the Related Art

Compound (II), (2S)-2-tert-butoxy-2-(4-(2,3-dihydropyrano[4,3,2-de]quinolin-7-yl)-2-methylquinolin-3-yl)acetic acid, is an HIV non-catalytic site integrase inhibitor.

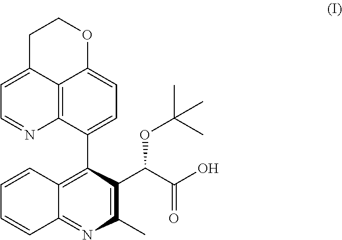

Compound (I) falls within the scope of the HIV inhibitors disclosed in WO 2007/131350. Compound (I) is disclosed specifically as compound no. 1144 in WO 2009/062285. Compound (I) can be prepared according to the general procedures found in WO 2007/131350 and WO 2009/062285, which are hereby incorporated by reference.

In drug development, it is necessary to produce a compound that can enable formulation to meet exacting pharmaceutical requirements and specifications. This is typically achieved through the use of a stable crystalline form of the drug. It is also desirable to produce a non-solvate stable crystalline form. When a drug exists as a solvate form, there is a need to control the amount of solvents in the drug form. It is desirable to select a drug form that is easily manufactured and may be produce on a large-scale in a cost-efficient manner. The present invention fulfills these needs and provides further related advantages.

### BRIEF SUMMARY

The present invention provides a novel salt form of Compound (I), novel crystalline forms of Compound (I) and novel crystalline forms of the hydrochloride salt of Compound (I) which are useful in the treatment of an HIV infection.

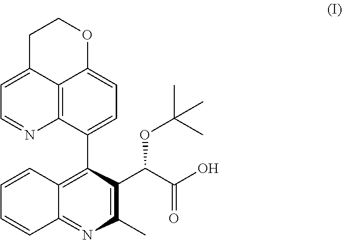

Further objects of this invention arise for the one skilled in the art from the following description and the examples.

In one embodiment, the invention is directed to a hydrochloride salt of Compound (I):

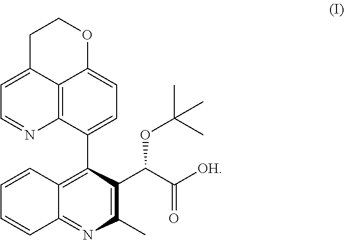

The above hydrochloride salt form of Compound (I) may be in a non-crystalline or crystalline state, and each of which may exist as a solvate or non-solvate. In one embodiment of the invention, the hydrochloride salt of Compound (I) is in crystalline form.

Another embodiment of the invention is a crystalline hydrochloride salt of Compound (I) in crystalline Type A having an X-ray powder diffraction pattern comprising peaks at 8.1, 9.3, 11.2, 28.4 and 28.6 degrees 2θ (±0.2 degrees 2θ) when measured using CuKα radiation.

Another embodiment of the invention is a crystalline hydrochloride salt of Compound (I) in crystalline Type A having an X-ray powder diffraction pattern comprising peaks at 8.1, 9.3, 11.2, 13.0, 28.4 and 28.6 degrees 2θ (±0.2 degrees 2θ) when measured using CuKα radiation.

Another embodiment of the invention is a crystalline hydrochloride salt of Compound (I) in crystalline Type A having an X-ray powder diffraction pattern comprising peaks at 8.1, 9.3, 11.2, 13.0, 28.4, 28.6, 10.4, 12.1, 18.8, 19.8, 22.1 and 22.4 degrees 2θ (±0.2 degrees 2θ) when measured using CuKα radiation.

Another embodiment of the invention is a crystalline hydrochloride salt of Compound (I) in crystalline Type A having an X-ray powder diffraction pattern substantially the same as that shown in FIG. 1.

Another embodiment of the invention is a crystalline hydrochloride salt of Compound (I) in crystalline Type A having a DSC thermal curve substantially the same as that shown in FIG. 2 indicated as Type A.

Another embodiment of the invention is a crystalline hydrochloride salt of Compound (I) in crystalline Type A having an X-ray powder diffraction pattern comprising peaks at 8.1, 9.3, 11.2, 28.4 and 28.6 degrees 2θ (±0.2 degrees 2θ) when measured using CuKα radiation and having a DSC thermal curve substantially the same as that shown in FIG. 2 indicated as Type A.

Another embodiment of the invention is a crystalline hydrochloride salt of Compound (I) in crystalline Type A having a ¹³C-ssNMR spectrum having chemical shift peaks at 146.7, 140.4, 136.9, 123.1, 121.4, and 21.8 ppm (each peak is ±0.2 ppm).

Another embodiment of the invention is a crystalline hydrochloride salt of Compound (I) in crystalline Type A having a ¹³C-ssNMR spectrum having chemical shift peaks at 171.0, 146.7, 140.4, 136.9, 123.1, 121.4, and 21.8 ppm (each peak is ±0.2 ppm).

Another embodiment of the invention is a crystalline hydrochloride salt of Compound (I) in crystalline Type A having a ¹³C-ssNMR spectrum having chemical shift peaks at 171.0, 158.7, 154.2, 150.5, 146.7, 140.4, 136.9, 123.1, 121.4, 28.7 and 21.8 ppm (each peak is ±0.2 ppm).

Another embodiment of the invention is a crystalline hydrochloride salt of Compound (I) in crystalline Type A having a ¹³C-ssNMR spectrum having chemical shift peaks at 171.0, 158.7, 154.2, 150.5, 146.7, 140.4, 136.9, 133.0, 129.8, 128.8, 125.8, 123.1, 121.4, 118.5, 115.9, 110.7, 78.1, 72.2, 65.2, 28.7 and 21.8 ppm (each peak is ±0.2 ppm).

Another embodiment of the invention is a crystalline hydrochloride salt of Compound (I) in crystalline Type A having an X-ray powder diffraction pattern comprising peaks at 8.1, 9.3, 11.2, 28.4 and 28.6 degrees 2θ (±0.2 degrees 2θ) when measured using CuKα radiation and a ¹³C-ssNMR spectrum having chemical shift peaks at 146.7, 140.4, 136.9, 123.1, 121.4, and 21.8 ppm (each peak is ±0.2 ppm).

Another embodiment of the invention is a crystalline hydrochloride salt of Compound (I) in crystalline Type A having a ¹³C-ssNMR spectrum substantially the same as that shown in FIG. 3.

Another embodiment of the invention is a crystalline hydrochloride salt of Compound (I) in crystalline Type B having an X-ray powder diffraction pattern comprising peaks at 7.2, 8.9 and 10.7 degrees 2θ (±0.2 degrees 2θ) when measured using CuKα radiation.

Another embodiment of the invention is a crystalline hydrochloride salt of Compound (I) in crystalline Type B having an X-ray powder diffraction pattern comprising peaks at 7.2, 8.9, 9.7, 10.7, 12.0 and 12.6 degrees 2θ (±0.2 degrees 2θ) when measured using CuKα radiation.

Another embodiment of the invention is a crystalline hydrochloride salt of Compound (I) in crystalline Type B having an X-ray powder diffraction pattern comprising peaks at 7.2, 8.9, 9.7, 10.7, 12.0, 12.6, 16.2, 16.8, 18.3 and 21.0 degrees 2θ (±0.2 degrees 2θ) when measured using CuKα radiation.

Another embodiment of the invention is a crystalline hydrochloride salt of Compound (I) in crystalline Type B having an X-ray powder diffraction pattern substantially the same as that shown in FIG. 4 indicated as Type B.

Another embodiment of the invention is a crystalline hydrochloride salt of Compound (I) in crystalline Type B having a DSC thermal curve substantially the same as that shown in FIG. 2 indicated as Type B.

Another embodiment of the invention is a crystalline hydrochloride salt of Compound (I) in crystalline Type B having an X-ray powder diffraction pattern comprising peaks at 7.2, 8.9 and 10.7 degrees 2θ (±0.2 degrees 2θ) when measured using CuKα radiation and having a DSC thermal curve substantially the same as that shown in FIG. 2 indicated as Type B.

Another embodiment of the invention is Compound (I) in crystalline form, either as a solvate or a non-solvate:

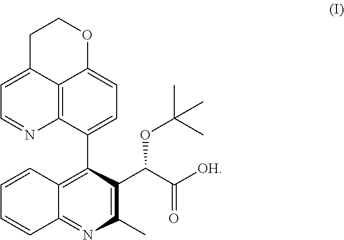

Another embodiment of the invention is crystalline Compound (I) in crystalline Form I having an X-ray powder diffraction pattern comprising a peak at 11.4 degrees 2θ (±0.2 degrees 2θ) when measured using CuKα radiation.

Another embodiment of the invention is crystalline Compound (I) in crystalline Form I having an X-ray powder diffraction pattern comprising peaks at 11.4 and 12.8 degrees 2θ (±0.2 degrees 2θ) when measured using CuKα radiation.

Another embodiment of the invention is crystalline Compound (I) in crystalline Form I having an X-ray powder diffraction pattern comprising peaks at 10.3, 11.4, 12.3, 12.8, 14.3, 18.9, 19.4, 19.8 and 21.6 degrees 2θ (±0.2 degrees 2θ) when measured using CuKα radiation.

Another embodiment of the invention is crystalline Compound (I) in crystalline Form I having an X-ray powder diffraction pattern substantially the same as that shown in FIG. 5.

Another embodiment of the invention is crystalline Compound (I) in crystalline Form I having a ¹³C-ssNMR spectrum having chemical shift peaks at 175.2, 155.8, 142.3, 135.5, 27.6 and 23.9 ppm (each peak is ±0.2 ppm).

Another embodiment of the invention is crystalline Compound (I) in crystalline Form I having a ¹³C-ssNMR spectrum having chemical shift peaks at 175.2, 158.5, 155.8, 150.5, 148.1, 147.9, 144.9, 142.3, 135.5, 27.6 and 23.9 ppm (each peak is ±0.2 ppm).

Another embodiment of the invention is crystalline Compound (I) in crystalline Form I having a ¹³C-ssNMR spectrum having chemical shift peaks at 175.2, 158.5, 155.8, 150.5, 148.1, 147.9, 144.9, 142.3, 135.5, 28.6, 27.6 and 23.9 ppm pm (each peak is ±0.2 ppm).

Another embodiment of the invention is crystalline Compound (I) in crystalline Form I having a ¹³C-ssNMR spectrum having chemical shift peaks at 175.2, 158.5, 155.8, 150.5, 148.1, 147.9, 144.9, 142.3, 135.5, 132.0, 131.0, 129.5, 129.2, 127.0, 118.6, 118.2, 110.7, 75.7, 71.6, 65.4, 28.6, 27.6 and 23.9 ppm (each peak is ±0.2 ppm).

Another embodiment of the invention is crystalline Compound (I) in crystalline Form I having an X-ray powder diffraction pattern comprising a peak at 11.4 degrees 2θ (±0.2 degrees 2θ) when measured using CuKα radiation and a ¹³C-ssNMR spectrum having chemical shift peaks at 175.2, 155.8, 142.3, 135.5, 27.6 and 23.9 ppm (each peak is ±0.2 ppm).

Another embodiment of the invention is crystalline Compound (I) in crystalline Form I having a ¹³C-ssNMR spectrum substantially the same as that shown in FIG. 6.

Another embodiment of the invention is crystalline Compound (I) in crystalline Form II having an X-ray powder diffraction pattern comprising peaks at 6.0, 6.7 and 13.5 degrees 2θ (±0.2 degrees 2θ) when measured using CuKα radiation.

Another embodiment of the invention is crystalline Compound (I) in crystalline Form II having an X-ray powder diffraction pattern comprising peaks at 6.0, 6.7, 10.5, 10.9, 13.5 and 16.7 degrees 2θ (±0.2 degrees 2θ) when measured using CuKα radiation.

Another embodiment of the invention is crystalline Compound (I) in crystalline Form II having an X-ray powder diffraction pattern comprising peaks at 6.0, 6.7, 10.5, 10.9, 12.5, 13.5, 16.7, 17.8, 19.8 and 21.8 degrees 2θ (±0.2 degrees 2θ) when measured using CuKα radiation.

Another embodiment of the invention is crystalline Compound (I) in crystalline Form II having an X-ray powder diffraction pattern substantially the same as that shown in FIG. 8.

Another embodiment of the invention is crystalline Compound (I) in crystalline Form II having a DSC thermal curve substantially the same as that shown in FIG. 9.

Another embodiment of the invention is crystalline Compound (I) in crystalline Form II having an X-ray powder diffraction pattern comprising peaks at 6.0, 6.7 and 13.5 degrees 2θ (±0.2 degrees 2θ) and having a DSC thermal curve substantially the same as that shown in FIG. 9.

Another embodiment of the invention is crystalline Compound (I) in crystalline Form III having an X-ray powder diffraction pattern comprising peaks at 5.0 and 16.4 degrees 2θ (±0.2 degrees 2θ) when measured using CuKα radiation.

Another embodiment of the invention is crystalline Compound (I) in crystalline Form III having an X-ray powder diffraction pattern comprising peaks at 5.0, 9.7, 10.0, 10.5, 10.9, 11.8, 12.2, 13.5, 13.8, 14.8, 15.6, 17.0, 17.6 and 19.8 degrees 2θ (±0.2 degrees 2θ) when measured using CuKα radiation.

Another embodiment of the invention is crystalline Compound (I) in crystalline Form III having an X-ray powder diffraction pattern substantially the same as that shown in FIG. 10.

Another embodiment of the invention is crystalline Compound (I) in crystalline Form III having a DSC thermal curve substantially the same as that shown in FIG. 11.

Another embodiment of the invention is crystalline Compound (I) in crystalline Form III having an X-ray powder diffraction pattern comprising peaks at 5.0 and 16.4 degrees 2θ (±0.2 degrees 2θ) and having a DSC thermal curve substantially the same as that shown in FIG. 11.

Another embodiment of the invention is crystalline Compound (I) in crystalline Form III having a ¹³C-ssNMR spectrum having chemical shift peaks at 173.1, 172.6, 161.5, 160.4, 152.3, 151.4, 145.8, 141.1, 123.9, 119.6, 30.3, 26.8 and 25.1 ppm (each peak is ±0.2 ppm).

Another embodiment of the invention is crystalline Compound (I) in crystalline Form III having a ¹³C-ssNMR spectrum having chemical shift peaks at 173.1, 172.6, 161.5, 160.4, 152.3, 151.4, 145.8, 141.1, 123.9, 119.6, 30.3, 26.8 and 25.1 ppm and further comprising chemical shift peaks at 171.1, 158.1, 156.2, 154.2, 150.0, 149.2, 148.5, 147.5, 147.0, 145.1 and 142.7 ppm (each peak is ±0.2 ppm).

Another embodiment of the invention is crystalline Compound (I) in crystalline Form III having a ¹³C-ssNMR spectrum having chemical shift peaks at 173.1, 172.6, 161.5, 160.4, 152.3, 151.4, 145.8, 141.1, 123.9, 119.6, 30.3, 26.8 and 25.1 ppm and further comprising chemical shift peaks at 171.1, 158.1, 156.2, 154.2, 150.0, 149.2, 148.5, 147.5, 147.0, 145.1, 142.7, 28.5 and 23.1 ppm (each peak is ±0.2 ppm).

Another embodiment of the invention is crystalline Compound (I) in crystalline Form III having a ¹³C-ssNMR spectrum having chemical shift peaks at 173.1, 172.6, 161.5, 160.4, 152.3, 151.4, 145.8, 141.1, 123.9, 119.6, 30.3, 26.8 and 25.1 ppm and further comprising chemical shift peaks at 171.1, 158.1, 156.2, 154.2, 150.0, 149.2, 148.0, 148.5, 147.5, 147.0, 145.1, 142.7, 136.4, 132.9, 131.9, 130.6, 129.8, 128.6, 127.7, 126.8, 126.1, 117.8, 117.4, 115.8, 110.7, 109.4, 75.8, 75.5, 74.2, 71.7, 69.8, 66.7, 28.5 and 23.1 ppm (each peak is ±0.2 ppm).

Another embodiment of the invention is crystalline Compound (I) in crystalline Form III having an X-ray powder diffraction pattern comprising peaks at 5.0 and 16.4 degrees 2θ (±0.2 degrees 2θ) when measured using CuKα radiation and a ¹³C-ssNMR spectrum having chemical shift peaks at 173.1, 172.6, 161.5, 160.4, 152.3, 151.4, 145.8, 141.1, 123.9, 119.6, 30.3, 26.8 and 25.1 ppm (each peak is ±0.2 ppm).

Another embodiment of the invention is crystalline Compound (I) in crystalline Form III having a ¹³C-ssNMR spectrum substantially the same as that shown in FIG. 11.

Another embodiment of the invention is a pharmaceutical composition comprising a hydrochloride salt of Compound (I) and at least one pharmaceutically acceptable carrier or diluent.

Another embodiment of the invention is a pharmaceutical composition comprising a a crystalline form of Compound (I), and at least one pharmaceutically acceptable carrier or diluent.

Another embodiment of the invention is a pharmaceutical composition as described above further comprising at least one other antiviral agent.

Another embodiment of the invention is the use of a pharmaceutical composition as described above for the treatment of an HIV infection in a human having or at risk of having the infection.

Another embodiment of the invention involves a method of treating or preventing an HIV infection in a human by administering to the human a therapeutically effective amount of Compound (I) in crystalline form as described above, or a composition as described above comprising Compound (I) in crystalline form, alone or in combination with at least one other antiviral agent, administered together or separately.

Another embodiment of the invention involves a method of treating or preventing an HIV infection in a human by administering to the human a therapeutically effective amount of a non-crystalline form of the hydrochloride salt of Compound (I), or a composition as described above comprising a non-crystalline form of the hydrochloride salt of Compound (I), alone or in combination with at least one other antiviral agent, administered together or separately.

Another embodiment of the invention involves a method of treating or preventing an HIV infection in a human by administering to the human a therapeutically effective amount of a crystalline form of the hydrochloride salt of Compound (I), or a composition as described above comprising a crystalline form of the hydrochloride salt of Compound (I), alone or in combination with at least one other antiviral agent, administered together or separately.

Also within the scope of this invention is the use of Compound (I) in crystalline form, as described herein, for the manufacture of a medicament for the treatment or prevention of an HIV infection in a human.

Also within the scope of this invention is the use of a non-crystalline form of the hydrochloride salt of Compound (I), as described herein, for the manufacture of a medicament for the treatment or prevention of an HIV infection in a human.

Also within the scope of this invention is the use of a crystalline form of the hydrochloride salt of Compound (I), as described herein, for the manufacture of a medicament for the treatment or prevention of an HIV infection in a human.

Another embodiment of this invention is a process to prepare crystalline form Type A of the hydrochloride salt of Compound (I) comprising the following steps:
(i) dissolving Compound (I) in a suitable solvent(s), and then adding an aqueous solution of HCl;
(ii) slowly heating the mixture in step (i) with stirring to a temperature to obtain a solution or slurry;
(iii) slowly cooling the mixture obtained in step (ii);
(iv) slowly adding an anti-solvent; and
(v) collecting the solid material obtained in step (iv) to obtain the hydrochloride salt of Compound (I), Type A.

Another embodiment of this invention is a process to prepare crystalline form Type A of the hydrochloride salt of Compound (I) comprising the following steps:
(a) dissolving Compound (I) in a suitable solvent at a temperature greater than room temperature and then polish-filtering;
(b) optionally, adjusting the solution volume;
(c) cooling the solution temperature;
(d) adding dilute HCl in water or an aliphatic alcohol;
(e) initiating crystallization by seeding with Type A crystals of the hydrochloride salt of Compound (I);
(f) continuing crystallization by controlled slow addition of dilute HCl in water or an aliphatic alcohol;
(g) crystallizing the product further out of solution with the addition of non-polar solvents; and
(h) filtering and drying to provide crystals of the hydrochloride salt of Compound (I) Type A.

Another embodiment of this invention is a process to prepare crystalline form Type B of the hydrochloride salt of Compound (I) comprising the following steps:
(i) dissolving Compound (I) in a suitable solvent(s), and then adding an aqueous solution of HCl;
(ii) removing the solvent;
(iii) adding a suitable crystallization solvent to the residue obtained in step (ii);
(iv) leaving the mixture from step (iii) to stand until crystals are formed; and
(v) isolating the precipitated crystals to obtain the hydrochloride salt of Compound (I), Type B.

Another embodiment of this invention is the process to prepare crystalline Compound (I), Form I comprising the following steps:
(i) dissolving Compound (I) in a suitable solvent(s) at room temperature;
(ii) stirring the mixture for a period of time;
(iii) slowly heating the mixture in step (ii) to a temperature to obtain a solution or slurry and holding the mixture at this temperature for a period of time;
(iv) slowly cooling the mixture obtained in step (iii);
(v) leaving the mixture from step (iii) to stand, with stirring, at room temperature until crystals are formed; and
(vi) isolating the crystals to obtain Compound (I), Form I with solvent(s).

Another embodiment of this invention is the process to prepare crystalline Compound (I), Form II comprising the following steps:
(i) dissolving Compound (I) in a suitable solvent(s) at room temperature;
(ii) slowly heating the mixture in step (i) to a temperature to obtain a solution;
(iii) slowly cooling the solution obtained in step (ii);
(iv) leaving the mixture from step (iii) to stand, with stirring, until crystals are formed; and
(v) isolating the precipitated crystals to obtain Compound (I), Form II with solvent(s).

Another embodiment of this invention is the process to prepare crystalline Compound (I), Form III comprising the following steps:
(i) slurrying Compound (I), Form II in water.
(ii) slowly heating the mixture to a temperature to obtain a slurry, and leaving the mixture to stand, with stirring, at this temperature for a period of time;
(iii) slowly cooling the slurry obtained in step (ii); and
(iv) isolating the crystals to obtain Compound (I), Form III.

As one of skill in the art will appreciate, in each of the foregoing synthetic processes, the recited steps may (i) occur individually or one or more steps may combined into a singe step, (ii) occur in the order recited or in an alternative order and (iii) occur optionally.

Further objects of this invention arise for the one skilled in the art from the following description and the examples.

### BRIEF DESCRIPTION OF THE DRAWINGS

FIG. 1 is the XRPD of the hydrochloride salt of Compound (I), Type A.

FIG. 2 is the DSC (Differential Scanning Calorimetry) of the hydrochloride salt of Compound (I), Type A and Type B.

FIG. 3 is the ¹³C-solid-state NMR spectrum of the hydrochloride salt of Compound (I). Type A.

FIG. 4 is the XRPD of crystalline Compound (I), Type A and Type B.

FIG. 5 is the XRPD of crystalline Compound (I), Form I.

FIG. 6 is the ¹³C-solid-state NMR spectrum of crystalline Compound (I), Form I.

FIG. 7 is the DSC of crystalline Compound (I), Form I obtained from different solvent systems.

FIG. 8 is the XRPD of crystalline Compound (I), Form II.

FIG. 9 is the DSC of crystalline Compound (I), Form II.

FIG. 10 is the XRPD of crystalline Compound (I), Form III.

FIG. 11 is the DSC of crystalline Compound (I), Form III.

FIG. 12 is the ¹³C-solid-state NMR spectrum of Compound (I), Form III.

### DETAILED DESCRIPTION

### Definitions

Terms not specifically defined herein should be given the meanings that would be given to them by one of skill in the art in light of the disclosure and the context. As used throughout the present application, however, unless specified to the contrary, the following terms have the meaning indicated:

### Compound (I), (2S)-2-tert-butoxy-2-(4-(2,3-dihydropyrano[4,3,2-de]quinolin-7-yl)-2-methylquinolin-3-yl)acetic acid

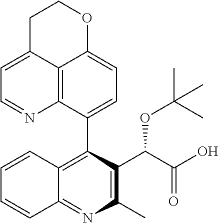

may alternatively be depicted as:

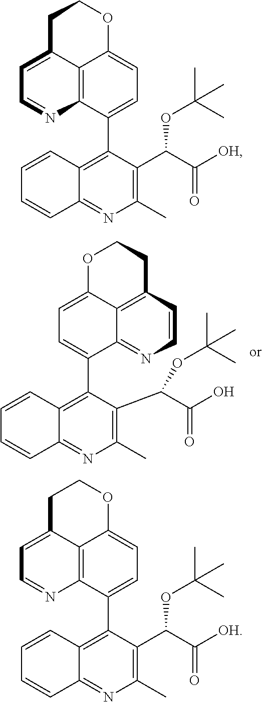

In addition, as one of skill in the art would appreciate, Compound (I) may alternatively be depicted in a zwitterionic form.

The term “solvate” refers to a crystalline solid containing amounts of a solvent incorporated within the crystal structure. As used herein, the term “solvate” includes hydrates.

The term “non-solvate” refers to a crystalline solid in which no solvent molecules occupy a specific crystallographic site.

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. For example, such salts include acetates, ascorbates, benzenesulfonates, benzoates, besylates, bicarbonates, bitartrates, bromides/hydrobromides, Ca-edetates/edetates, camsylates, carbonates, chlorides/hydrochlorides, citrates, edisylates, ethane disulfonates, estolates esylates, fumarates, gluceptates, gluconates, glutamates, glycolates, glycollylarsnilates, hexylresorcinates, hydrabamines, hydroxymaleates, hydroxynaphthoates, iodides, isothionates, lactates, lactobionates, malates, maleates, mandelates, methanesulfonates, mesylates, methylbromides, methylnitrates, methylsulfates, mucates, napsylates, nitrates, oxalates, pamoates, pantothenates, phenylacetates, phosphates/diphosphates, polygalacturonates, propionates, salicylates, stearates subacetates, succinates, sulfamides, sulfates, tannates, tartrates, teoclates, toluenesulfonates, triethiodides, ammonium, benzathines, chloroprocaines, cholines, diethanolamines, ethylenediamines, meglumines and procaines. Further pharmaceutically acceptable salts can be formed with cations from metals like aluminium, calcium, lithium, magnesium, potassium, sodium, zinc and the like. (also see Pharmaceutical salts, Birge, S. M. et al., J. Pharm. Sci., (1977), 66, 1-19).

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention (e.g., trifluoro acetate salts) also comprise a part of the invention.

The term “treating” with respect to the treatment of a disease-state in a patient include (i) inhibiting or ameliorating the disease-state in a patient, e.g., arresting or slowing its development; or (ii) relieving the disease-state in a patient, i.e., causing regression or cure of the disease-state. In the case of HIV, treatment includes reducing the level of HIV viral load in a patient.

The term “antiviral agent” as used herein is intended to mean an agent that is effective to inhibit the formation and/or replication of a virus in a human, including but not limited to agents that interfere with either host or viral mechanisms necessary for the formation and/or replication of a virus in a human. The term “antiviral agent” includes, for example, an HIV integrase catalytic site inhibitor selected from the group consisting: raltegravir (ISENTRESS®; Merck); elvitegravir (Gilead); soltegravir (GSK; ViiV); and GSK 1265744 (GSK; ViiV); an HIV nucleoside reverse transcriptase inhibitor selected from the group consisting of: abacavir (ZIAGEN®; GSK); didanosine (VIDEX®; BMS); tenofovir (VIREAD®; Gilead); emtricitabine (EMTRIVA®; Gilead); lamivudine (EPIVIR®; GSK/Shire); stavudine (ZERIT®; BMS); zidovudine (RETROVIR®; GSK); elvucitabine (Achillion); and festinavir (Oncolys); an HIV non-nucleoside reverse transcriptase inhibitor selected from the group consisting of: nevirapine (VIRAMUNE®; BI); efavirenz (SUSTIVA®; BMS); etravirine (INTELENCE®; J&J); rilpivirine (TMC278, R278474; J&J); fosdevirine (GSKN/ViiV); and lersivirine (Pfizer/ViiV); an HIV protease inhibitor selected from the group consisting of: atazanavir (REYATAZ®; BMS); darunavir (PREZISTA®; J&J); indinavir (CRIXIVAN®; Merck); lopinavir (KELETRA®; Abbott); nelfinavir (VIRACEPT®; Pfizer); saquinavir (INVIRASE®; Hoffmann-LaRoche); tipranavir (APTIVUS®; BI); ritonavir (NORVIR®; Abbott); and fosamprenavir (LEXIVA®; GSK/Vertex); an HIV entry inhibitor selected from: maraviroc (SELZENTRY®; Pfizer); enfuvirtide (FUZEON®; Trimeris); and BMS-663068 (BMS); and an HIV maturation inhibitor selected from: bevirimat (Myriad Genetics).

Hydrochloride Salt of Compound (I)

The hydrochloride salt of Compound (I) can be isolated in a non-crystalline form, a crystalline form or a mixture of both. The non-crystalline or crystalline forms may exist as a solvate or non-solvate.

The hydrochloride salt of Compound (I) can be isolated as crystalline polymorphic forms, including crystalline polymorphic forms designated herein as “Type A” and “Type B”.

Type A

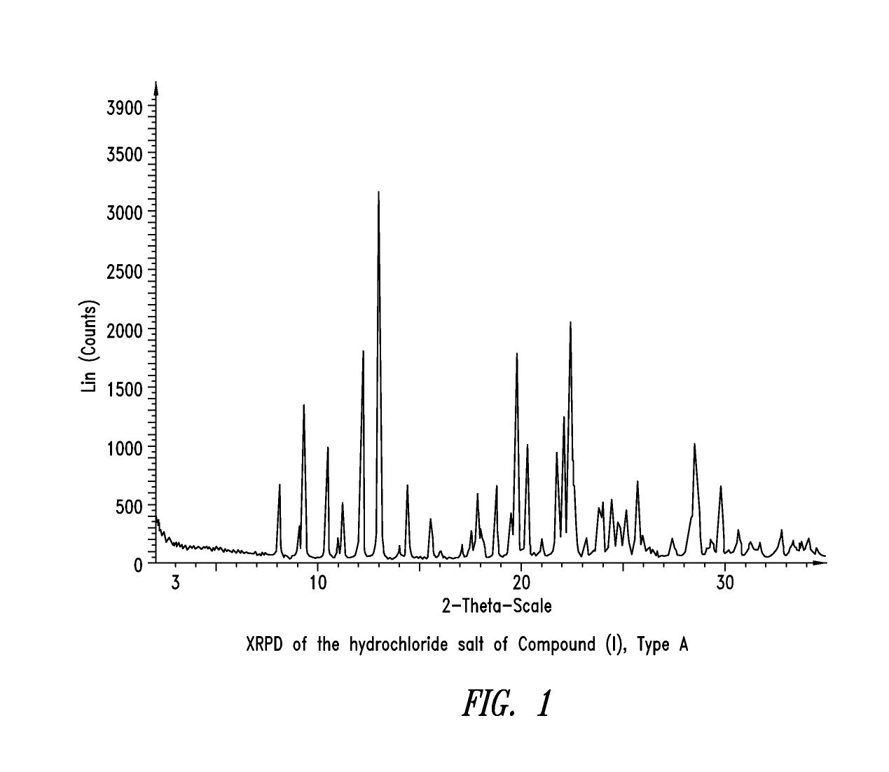

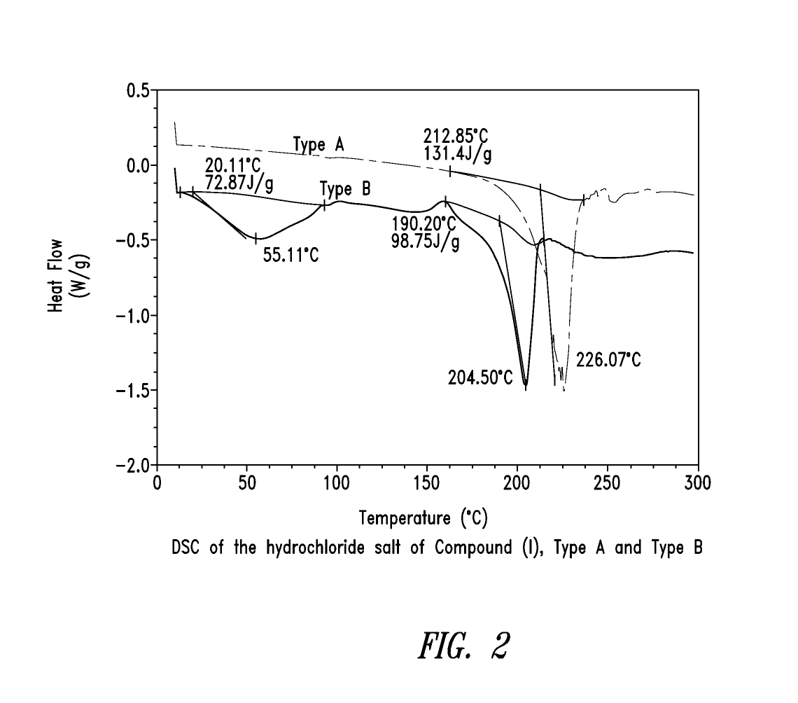

Type A is a non-solvate crystalline form of the hydrochloride salt of Compound (I). Type A is thermally stable with minimal weight loss during heating up to 200° C. Type A is non-hygroscopic based on moisture sorption/desorption measurements. Type A exhibits physical and chemical stability under stress conditions. Type A has solubility greater than 24 mg/ml at pH 2, 4.5 and 6.8, and has an intrinsic dissolution rate of 4528 μg/[cm²×min] in a pH 2.0 buffer. The XRPD pattern of Type A is shown in FIG. 1. The characteristic peak positions and relative intensities for the XRPD pattern in FIG. 1 for Type A is shown in Table 1:

Hydrochloride Salt of Compound (I),
Type A

| Angle | |
|---|---|
| 2-Theta ° | Relative Intensity % |
| 8.1 | 21 |
| 9.3 | 41 |
| 10.4 | 32 |
| 11.2 | 15 |
| 12.1 | 58 |
| 13.0 | 100 |
| 18.8 | 22 |
| 19.8 | 56 |
| 22.1 | 40 |
| 22.4 | 65 |
| 28.4 | 12 |
| 28.6 | 32 |

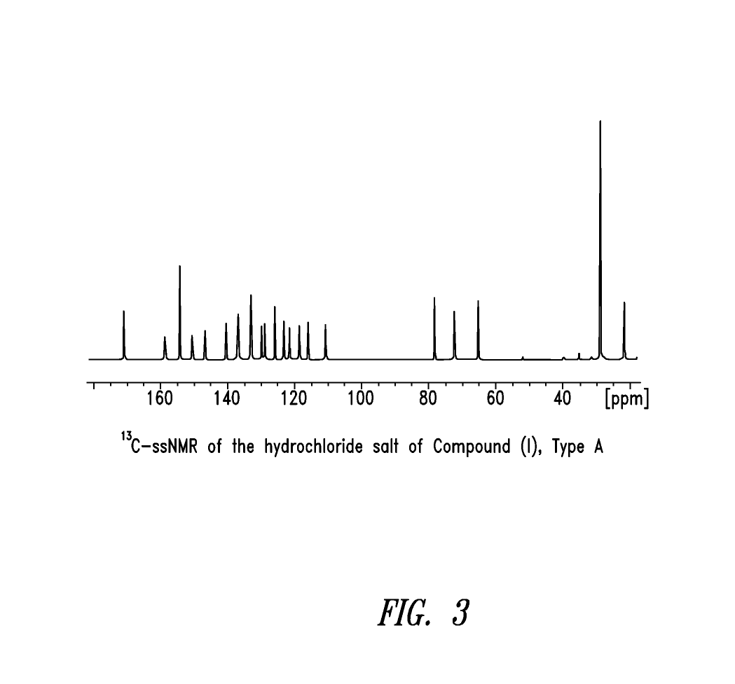

FIG. 2 shows the DSC thermal curve for Type A crystals where the DSC is performed at a heating rate of 10° C. per minute in a crimped cup.

An embodiment of the invention is directed to a crystalline polymorph of a hydrochloride salt of Compound (I), Type A, having an X-ray powder diffraction pattern (XRPD) comprising peaks at 8.1, 9.3, 11.2, 28.4 and 28.6 degrees 2θ (±0.2 degrees 2θ) when measured using CuKα radiation.

Another embodiment is directed to a crystalline polymorph of a hydrochloride salt of Compound (I), Type A, having an XRPD pattern comprising peaks at 8.1, 9.3, 11.2, 28.4 and 28.6 degrees 2θ (±0.2 degrees 2θ) as described above and further comprising a peak at 13.0 degrees 2θ (±0.2 degrees 2θ) when measured using CuKα radiation.

Another embodiment is directed to a crystalline polymorph of a hydrochloride salt of Compound (I), Type A, having an XRPD pattern comprising peaks at 8.1, 9.3, 11.2, 28.4 and 28.6 degrees 2θ (±0.2 degrees 2θ) as described above and further comprising peaks at 10.4, 12.1, 13.0, 18.8, 19.8, 22.1 and 22.4 degrees 2θ (±0.2 degrees 2θ) when measured using CuKα radiation.

Another embodiment is directed to a crystalline polymorph of a hydrochloride salt of Compound (I), Type A, exhibiting an XRPD pattern substantially the same as that shown in FIG. 1.

Another embodiment is directed to a crystalline polymorph of a hydrochloride salt of Compound (I), Type A, having a DSC thermal curve substantially the same as that shown in FIG. 2 indicated as Type A.

Another embodiment is directed to a crystalline polymorph of a hydrochloride salt of Compound (I), Type A, having an XRPD pattern comprising peaks at 8.1, 9.3, 11.2, 28.4 and 28.6 degrees 2θ (±0.2 degrees 2θ) as described above and also exhibiting a DSC thermal curve substantially the same as that shown in FIG. 2 indicated as Type A.

FIG. 3 is the ¹³C-solid-state Nuclear Magnetic Resonance (ssNMR) spectrum of crystalline polymorph of Compound (I), Type A.

An embodiment of the invention is directed to a crystalline polymorph of a hydrochloride salt of Compound (I), Type A, having a ¹³C-ssNMR spectrum having chemical shift peaks at 146.7, 140.4, 136.9, 123.1, 121.4, and 21.8 ppm (each peak is ±0.2 ppm).

Another embodiment of the invention is directed to a crystalline polymorph of a hydrochloride salt of Compound (I), Type A, having a ¹³C-ssNMR spectrum having chemical shift peaks at 148.7, 140.4, 136.9, 123.1, 121.4, and 21.8 ppm and further comprising a chemical shift peak at 171.0 ppm (each peak is ±0.2 ppm).

Another embodiment of the invention is directed to a crystalline polymorph of a hydrochloride salt of Compound (I), Type A, having a ¹³C-ssNMR spectrum having chemical shift peaks at 146.7, 140.4, 136.9, 123.1, 121.4, and 21.8 ppm and further comprising chemical shift peaks at 171.0, 158.7, 154.2, 150.5 and 28.7 ppm (each peak is ±0.2 ppm).

Another embodiment of the invention is directed to a crystalline polymorph of a hydrochloride salt of Compound (I), Type A, having a ¹³C-ssNMR spectrum having chemical shift peaks at 146.7, 140.4, 136.9, 123.1, 121.4, and 21.8 ppm (each peak is ±0.2 ppm) and further comprising chemical shift peaks at 171.0, 158.7, 154.2, 150.5, 133.0, 129.8, 128.8, 125.8, 118.5, 115.9, 110.7, 78.1, 72.2, 65.2 and 28.7 ppm (each peak is ±0.2 ppm).

Another embodiment is directed to a crystalline polymorph of a hydrochloride salt of Compound (I), Type A, having an XRPD pattern comprising peaks at 8.1, 9.3, 11.2, 28.4 and 28.6 degrees 2θ (±0.2 degrees 2θ) as described above or a ¹³C-ssNMR spectrum having chemical shift peaks at 146.7, 140.4, 136.9, 123.1, 121.4, and 21.8 ppm (each peak is ±0.2 ppm).

Another embodiment is directed to a crystalline polymorph of a hydrochloride salt of Compound (I), Type A, having an XRPD pattern comprising peaks at 8.1, 9.3, 11.2, 28.4 and 28.6 degrees 2θ (±0.2 degrees 2θ) as described above and also a ¹³C-ssNMR spectrum having chemical shift peaks at 146.7, 140.4, 136.9, 123.1, 121.4, and 21.8 ppm (each peak is ±0.2 ppm).

Another embodiment is directed to a crystalline polymorph of a hydrochloride salt of Compound (I), Type A, exhibiting a ¹³C-ssNMR spectrum substantially the same as that shown in FIG. 3.

Another embodiment is directed to a quantity of a hydrochloride salt of Compound (I) wherein at least 50%, preferably at least 75%, more preferably at least 95%, more preferably at least 99%, more preferably 100%, of said substance is present in crystalline form, for example, in the form of the Type A crystalline polymorph as characterized by any of the abovementioned XRPD or ¹³C-ssNMR spectra defined embodiments. The presence of such amounts of Type A in a quantity of a hydrochloride salt of Compound (I) is typically measurable using XRPD analysis of the compound.

An additional embodiment is directed to a pharmaceutical composition comprising a hydrochloride salt of Compound (I) and a pharmaceutically acceptable carrier or diluent, wherein at least 50%, preferably at least 75%, more preferably at least 95%, more preferably at least 99%, more preferably 100%, of said hydrochloride salt of Compound (I) in the composition is present in crystalline form, for example, in the form of the Type A crystalline polymorph as characterized by any of the abovementioned XRPD or ¹³C-ssNMR spectrum defined embodiments.

An additional embodiment is directed to a pharmaceutical composition comprising a hydrochloride salt of Compound (I) and a pharmaceutically acceptable carrier or diluent and further comprising at least one other antiviral agent, wherein at least 50%, preferably at least 75%, more preferably at least 95%, more preferably at least 99%, more preferably 100%, of said hydrochloride salt of Compound (I) in the composition is present in crystalline form, for example, in the form of the Type A crystalline polymorph as characterized by any of the abovementioned XRPD or ¹³C-ssNMR spectrum defined embodiments.

The present invention provides a process for the preparation of Type A which comprises crystallizing a hydrochloride salt of Compound (I) from a solution in solvents under conditions which yield Type A. The precise conditions under which Type A is formed may be empirically determined and it is only possible to give methods which have been found to be suitable in practice. As one of skill in the art will appreciate, in each of the following synthetic processes, the recited steps may (i) occur individually or one or more steps may combined into a singe step, (ii) occur in the order recited or in an alternative order and (iii) occur optionally.

It has been found that Type A of the hydrochloride salt of Compound (I) may be prepared by a process comprising the following steps, which process is also an embodiment of the present invention:
(i) dissolving Compound (I) in a suitable solvent(s), and then adding an aqueous solution of HCl;
(ii) slowly heating the mixture in step (i) with stirring to a temperature to obtain a solution or slurry;
(iii) slowly cooling the mixture obtained in step (ii), for example at a rate of 5° C./hour;
(iv) slowly adding an anti-solvent, such as, for example, isopropanol; and
(v) collecting the solid material obtained in step (iv) to obtain the hydrochloride salt of Compound (I), Type A.

In step (i), a suitable solvent that may be employed in this process includes an aliphatic alcohol, for example, ethanol (e.g., denatured, 200 proof or 100% pure), methyl ethyl ketone, tetrahydrofuran, acetonitrile, dichloroethane, methyl-t-butyl-ether or water.

The resulting crystals of Type A may be recovered by any conventional methods known in the art.

In the final step (v), the resulting solids obtained in step (iv) may be collected and dried at high temperature using conventional collection and high-temperature drying techniques, for example, filtration and vacuum oven.

It has been found that Type A of the hydrochloride salt of Compound (I) may alternatively be prepared by a process comprising the following steps, which process is also an embodiment of the present invention:
(a) dissolving Compound (I) in a suitable solvent at a temperature greater than room temperature and then polish-filtering;
(b) optionally, adjusting the solution volume, such as, for example, adjusting the solution volume to 50-75% of the original volume;
(c) cooling the solution temperature, such as, for example, lowering the temperature by approximately 10-40° C., preferably the temperature after cooling is approximately 40-60° C.;
(d) adding dilute HCl in water or an aliphatic alcohol, such as isopropyl alcohol or ethyl alcohol, preferably isopropyl alcohol;
(e) initiating crystallization by seeding with Type A crystals of the hydrochloride salt of Compound (I);
(f) continuing crystallization by controlled slow addition of dilute HCl in water or an aliphatic alcohol, such as isopropyl alcohol or ethyl alcohol, preferably isopropyl alcohol, until, for example, approximately 1 equivalent is added;
(g) crystallizing the product further out of solution with the addition of non-polar solvents, such as heptanes, hexane, cyclohexane, and other antisolvents such as methyl-t-butyl ether, butyl acetate, preferably heptanes; and
(h) filtering and drying to provide crystals of the hydrochloride salt of Compound (I) Type A.

In step (a), a suitable solvent may be an aliphatic alcohol, preferably ethyl alcohol or isopropyl alcohol, more preferably ethyl alcohol. The temperature which is greater than room temperature, may be, for example 50-90° C., preferably 65-85° C., more preferably 75-80° C.

The resulting crystals of Type A may be recovered by any conventional methods known in the art.

In the final step (h), the resulting solids obtained in step (g) may be collected and dried at high temperature using conventional collection and high-temperature drying techniques, for example, filtration and vacuum oven. The process steps may of course be facilitated by conventional agitation techniques, e.g., stirring, and other conventional techniques as would be well understood for facilitation the process.

The process steps may of course be facilitated by conventional agitation techniques, e.g., stirring, and other conventional techniques as would be well understood for facilitation the process.

Type B

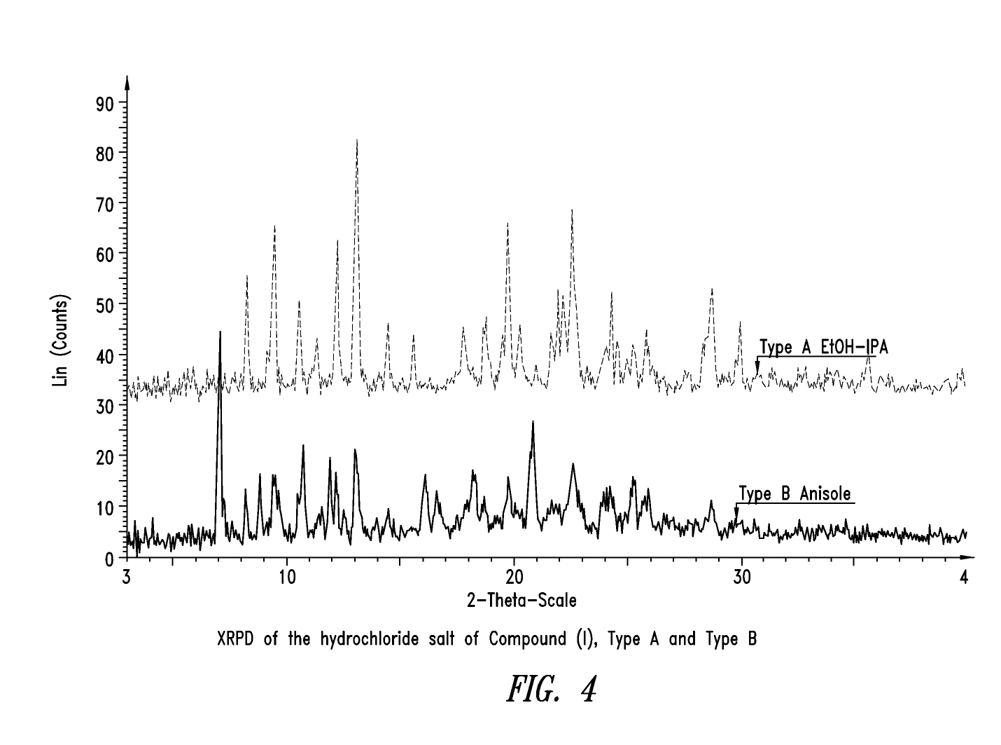

Type B is a solvate crystalline form of the hydrochloride salt of Compound (I). The XRPD pattern of Compound (I), Type B is shown in FIG. 4. The characteristic peak positions and relative intensities for the XRPD pattern in FIG. 4 for Type B are shown in Table 2:

Compound (I), Type B

| Angle | |
|---|---|
| 2-Theta ° | Relative Intensity % |
| 7.2 | 100 |
| 8.9 | 54 |
| 9.7 | 37 |
| 10.7 | 62 |
| 12.0 | 49 |
| 12.6 | 37 |
| 16.2 | 58 |
| 16.8 | 42 |
| 18.3 | 59 |
| 21.0 | 71 |

FIG. 2 shows the DSC thermal curve for Type B crystals where the DSC is performed at a heating rate of 10° C. per minute in a crimped cup.

An embodiment of the invention is directed to a crystalline polymorph of Compound (I), Type B, having an X-ray powder diffraction pattern comprising peaks at 7.2, 8.9 and 10.7 degrees 2θ (±0.2 degrees 2θ) when measured using CuKα radiation.

Another embodiment is directed to a crystalline polymorph of Compound (I), Type B, having an XRPD pattern comprising peaks at 7.2, 8.9 and 10.7 degrees 2θ (±0.2 degrees 2θ) and further comprising peaks at 9.7, 12.0 and 12.6 degrees 2θ (±0.2 degrees 2θ) when measured using CuKα radiation.

Another embodiment is directed to a crystalline polymorph of Compound (I), Type B, having an XRPD pattern comprising peaks at 7.2, 8.9 and 10.7 degrees 2θ (±0.2 degrees 2θ) and further comprising peaks at 9.7, 12.0, 12.6, 16.2, 16.8, 18.3 and 21.0 degrees 2θ (±0.2 degrees 2θ) when measured using CuKα radiation.

Another embodiment is directed to a crystalline polymorph of Compound (I), Type B, having an XRPD pattern substantially the same as that shown in FIG. 4.

An embodiment of the invention is directed to a crystalline polymorph of a hydrochloride salt of Compound (I), Type B, exhibiting a DSC thermal curve substantially the same as that shown in FIG. 2 indicated as Type B.

Another embodiment is directed to a crystalline polymorph of a hydrochloride salt of Compound (I), Type B, having an XRPD pattern comprising peaks at 7.2, 8.9 and 10.7 degrees 2θ (±0.2 degrees 2θ) as described above and also exhibiting a DSC thermal curve substantially the same as that shown in FIG. 2 indicated as Type B.

Another embodiment is directed to a quantity of a hydrochloride salt of Compound (I) wherein at least 50%, preferably at least 75%, more preferably at least 95%, more preferably at least 99%, more preferably 100%, of said substance is present in crystalline form, for example, in the form of the Type B crystalline polymorph as characterized by any of the abovementioned XRPD-defined embodiments. The presence of such amounts of Type B in a quantity of a hydrochloride salt of Compound (I) is typically measurable using XRPD analysis of the compound.

An additional embodiment is directed to a pharmaceutical composition comprising a hydrochloride salt of Compound (I) and a pharmaceutically acceptable carrier or diluent, wherein at least 50%, preferably at least 75%, more preferably at least 95%, more preferably at least 99%, more preferably 100%, of said hydrochloride salt of Compound (I) in the composition is present in crystalline form, for example, in the form of the Type B crystalline polymorph as defined above.

An additional embodiment is directed to a pharmaceutical composition comprising a hydrochloride salt of Compound (I) and a pharmaceutically acceptable carrier or diluent and further comprising at least one other antiviral agent, wherein at least 50%, preferably at least 75%, more preferably at least 95%, more preferably at least 99%, more preferably 100%, of said hydrochloride salt of Compound (I) in the composition is present in crystalline form, for example, in the form of the Type B crystalline polymorph as defined above.

The present invention provides a process for the preparation of Type B which comprises crystallizing a hydrochloride salt of Compound (I) from a solution in solvents under conditions which yield Type B. The precise conditions under which Type B is formed may be empirically determined and it is only possible to give methods which have been found to be suitable in practice. As one of skill in the art will appreciate, in each of the following synthetic processes, the recited steps may (i) occur individually or one or more steps may combined into a singe step, (ii) occur in the order recited or in an alternative order and (iii) occur optionally.

It has been found that Type B of the hydrochloride salt of Compound (I) may be prepared by a process comprising the following steps, which process is also an embodiment of the present invention:
(i) dissolving Compound (I) in a suitable solvent(s), and then adding an aqueous solution of HCl;
(ii) removing the solvent;
(iii) adding a suitable crystallization solvent to the residue obtained in step (ii);
(iv) leaving the mixture from step (iii) to stand until crystals are formed: and
(v) isolating the precipitated crystals to obtain the hydrochloride salt of Compound (I), Type B.

In step (i), a suitable solvent that may be employed in this process includes, for example, toluene or anisole.

The resulting crystals of Type B may be recovered by any conventional methods known in the art.

In the final step (v), the resulting solids obtained in step (iv) may be collected and dried at high temperature using conventional collection and high-temperature drying techniques, for example, filtration and vacuum oven.

The process steps may of course be facilitated by conventional agitation techniques, e.g., stirring, and other conventional techniques as would be well understood for facilitation of the process.

Compound (I)—Crystalline Polymorph Forms

Compound (I) can be isolated in a non-crystalline form, a crystalline form or a mixture of both. The non-crystalline or crystalline forms may exist as a solvate or non-solvate.

Compound (I) can be isolated as crystalline polymorphic forms, including crystalline polymorphic forms designated herein as “Form I”. “Form II” and “Form III”.

Compound (I), Form I

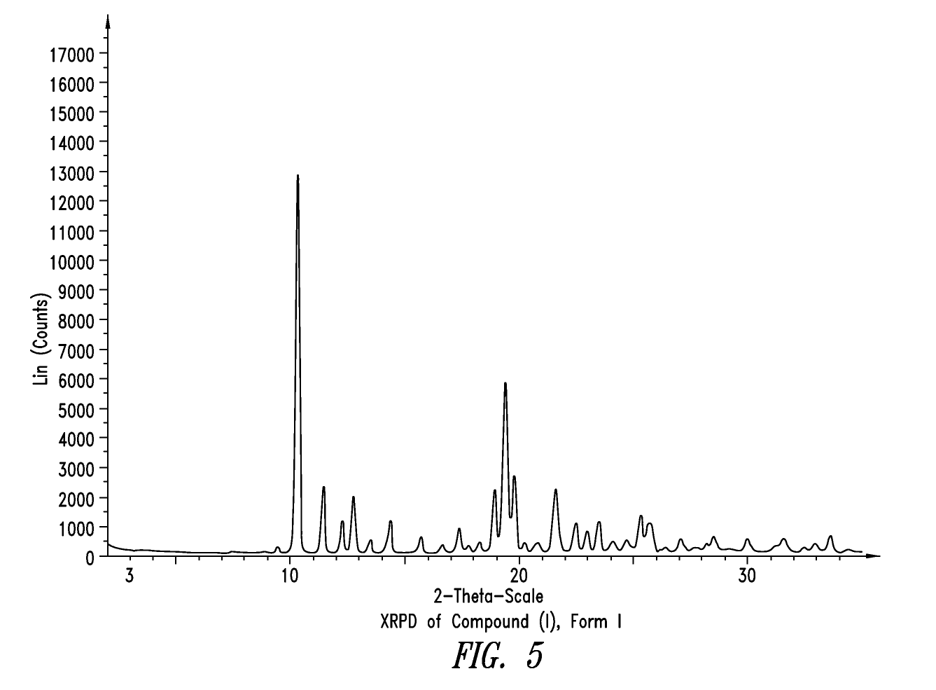

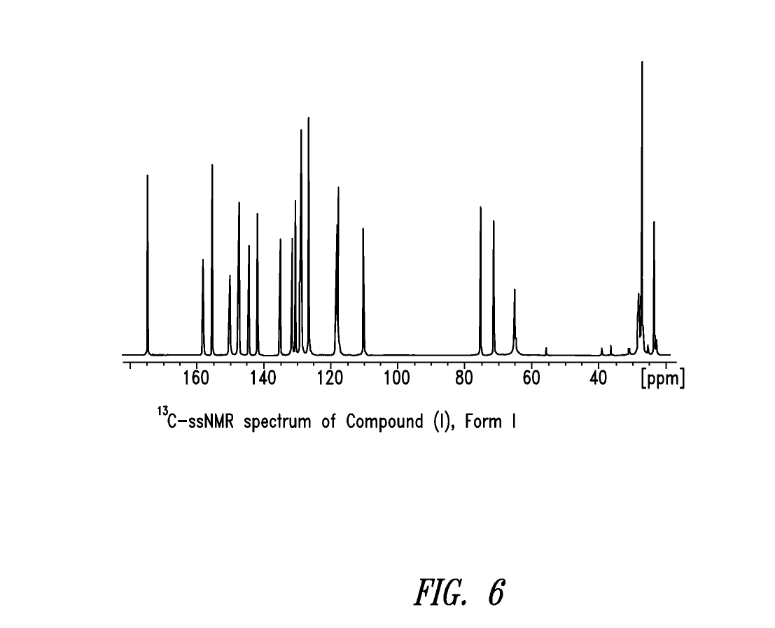

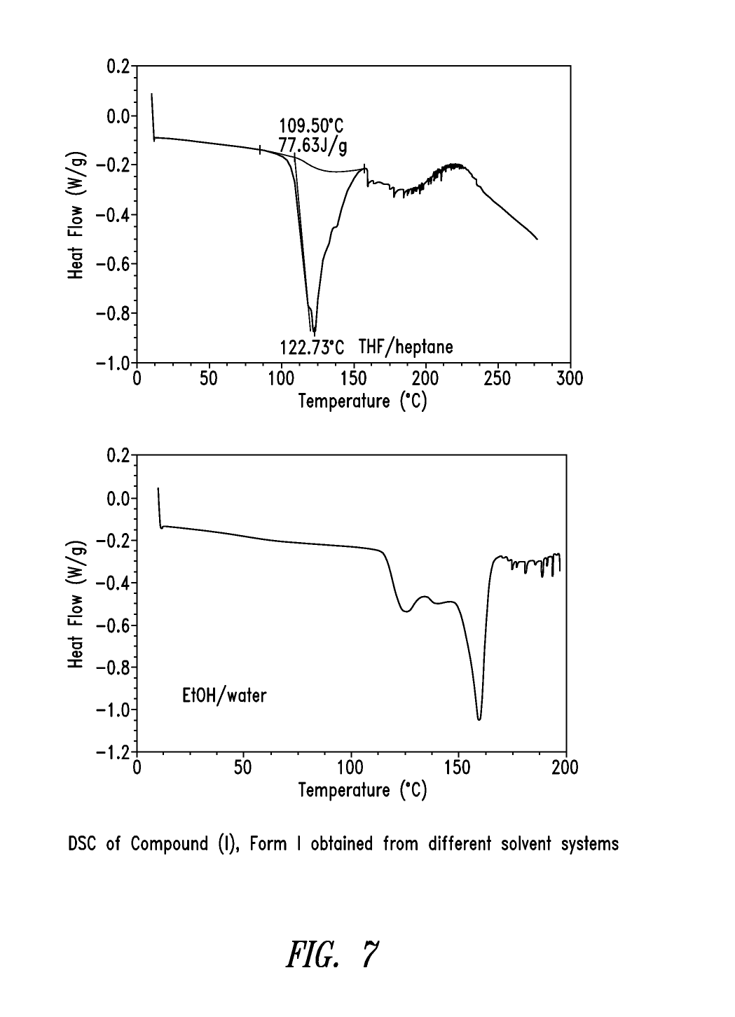

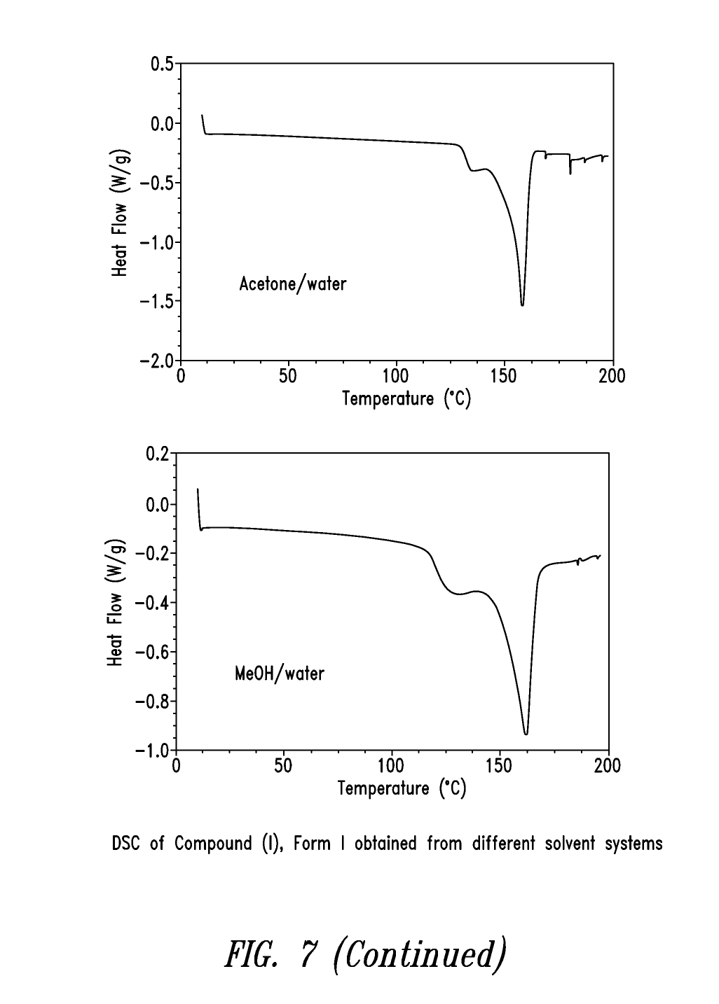

The XRPD pattern of Compound (I), Form I is shown in FIG. 5. Form I is a crystalline form with solvent encapsulation and the encapsulated solvent cannot be removed until melting. The solvent is unable to be removed through conventional techniques, such as, for example, drying, water vapor diffusion and hot slurry methods. Form I solvates, when generated from different solvent systems have different DSC profiles, as shown in FIG. 7. The encapsulation of solvent in Form I is confirmed by single crystal X-ray diffraction. The characteristic peak positions and relative intensities for the XRPD pattern in FIG. 5 for Form I are shown in Table 3:

Compound (I), Form I

| Angle | |
|---|---|
| 2-Theta ° | Relative Intensity % |
| 10.3 | 100 |
| 11.4 | 17 |
| 12.3 | 10 |
| 12.8 | 16 |
| 14.3 | 10 |
| 18.9 | 19 |
| 19.4 | 46 |
| 19.8 | 21 |
| 21.6 | 18 |

An embodiment of the invention is directed to a crystalline polymorph of Compound (I), Form I, having an X-ray powder diffraction pattern comprising a peak at 11.4 degrees 2θ (±0.2 degrees 2θ) when measured using CuKα radiation.

Another embodiment is directed to a crystalline polymorph of Compound (I), Form I, having an XRPD pattern comprising a peak at 11.4 degrees 2θ (±0.2 degrees 2θ) as described above and further comprising a peak at 12.8 degrees 2θ (±0.2 degrees 2θ) when measured using CuKα radiation.

Another embodiment is directed to a crystalline polymorph of Compound (I), Form I, having an XRPD pattern comprising a peak at 11.4 degrees 2θ (±0.2 degrees 2θ) as described above and further comprising peaks at 10.3, 12.3, 12.8, 14.3, 18.9, 19.4, 19.8 and 21.6 degrees 2θ (±0.2 degrees 2θ) when measured using CuKα radiation.

Another embodiment is directed to a crystalline polymorph of Compound (I), Form I, exhibiting an XRPD pattern substantially the same as that shown in FIG. 5.

FIG. 6 is the ¹³C-solid-state Nuclear Magnetic Resonance (ssNMR) spectrum of crystalline polymorph of Compound (I), Form I.

An embodiment of the invention is directed to a crystalline polymorph of Compound (I), Form I, having a ¹³C-ssNMR spectrum having chemical shift peaks at 175.2, 155.8, 142.3, 135.5, 27.6 and 23.9 ppm (each peak is ±0.2 ppm).

Another embodiment of the invention is directed to a crystalline polymorph of Compound (I), Form I, having a ¹³C-ssNMR spectrum having chemical shift peaks at 175.2, 155.8, 142.3, 135.5, 27.6 and 23.9 ppm and further comprising chemical shift peaks at 158.5, 150.5, 148.1, 147.9 and 144.9 ppm (each peak is ±0.2 ppm).

Another embodiment of the invention is directed to a crystalline polymorph of Compound (I), Form I, having a ¹³C-ssNMR spectrum having chemical shift peaks at 175.2, 155.8, 142.3, 135.5, 27.6 and 23.9 ppm and further comprising chemical shift peaks at 158.5, 150.5, 148.1, 147.9, 144.9 and 28.6 ppm (each peak is ±0.2 ppm).

Another embodiment of the invention is directed to a crystalline polymorph of Compound (I), Form I, having a ¹³C-ssNMR spectrum having chemical shift peaks at 175.2, 155.8, 142.3, 135.5, 27.6 and 23.9 ppm and further comprising chemical shift peaks at 158.5, 150.5, 148.1, 147.9, 144.9, 132.0, 131.0, 129.5, 129.2, 127.0, 118.6, 118.2, 110.7, 75.7, 71.6, 65.4 and 28.6 ppm (each peak is ±0.2 ppm).

Another embodiment is directed to a crystalline polymorph of Compound (I), Form I, having an XRPD pattern comprising a peak at 11.4 degrees 2θ (±0.2 degrees 2θ) as described above or a ¹³C-ssNMR spectrum having chemical shift peaks at 175.2, 155.8, 142.3, 135.5, 27.6 and 23.9 ppm (each peak is ±0.2 ppm).

Another embodiment is directed to a crystalline polymorph of Compound (I), Form I, having an XRPD pattern comprising a peak at 11.4 degrees 2θ (±0.2 degrees 2θ) as described above and also a ¹³C-ssNMR spectrum having chemical shift peaks at 175.2, 155.8, 142.3, 135.5, 27.6 and 23.9 ppm (each peak is ±0.2 ppm).

Another embodiment is directed to a crystalline polymorph of Compound (I), Form I, exhibiting a ¹³C-ssNMR spectrum substantially the same as that shown in FIG. 6.

Another embodiment is directed to a quantity of Compound (I) wherein at least 50%, preferably at least 75%, more preferably at least 95%, more preferably at least 99%, more preferably 100%, of said substance is present in crystalline form, for example, in the form of Form I crystalline polymorph as characterized by any of the abovementioned XRPD or ¹³C-ssNMR defined embodiments. The presence of such amounts of Form I in a quantity of Compound (I) is typically measurable using XRPD analysis of the compound.

An additional embodiment is directed to a pharmaceutical composition comprising Compound (I) and a pharmaceutically acceptable carrier or diluent, wherein at least 50%, preferably at least 75%, more preferably at least 95%, more preferably at least 99%, more preferably 100%, of the Compound (I) in the composition is present in crystalline form, for example, in the form of Form I crystalline polymorph as characterized by any of the abovementioned XRPD or ¹³C-ssNMR defined embodiments.

An additional embodiment is directed to a pharmaceutical composition comprising Compound (I) and a pharmaceutically acceptable carrier or diluent and further comprising at least one other antiviral agent, wherein at least 50%, preferably at least 75%, more preferably at least 95%, more preferably at least 99%, more preferably 100%, of the Compound (I) in the composition is present in crystalline form, for example, in the form of the Form I crystalline polymorph as characterized by any of the abovementioned XRPD-defined embodiments.

The present invention provides a process for the preparation of Form I which comprises crystallizing Compound (I) from a solution in solvents under conditions which yield Form I. The precise conditions under which Form I is formed may be empirically determined and it is only possible to give methods which have been found to be suitable in practice. As one of skill in the art will appreciate, in each of the following synthetic processes, the recited steps may (i) occur individually or one or more steps may combined into a singe step, (ii) occur in the order recited or in an alternative order and (iii) occur optionally.

It has been found that Compound (I), Form I may be prepared by a process comprising the following steps, which process is also an embodiment of the present invention:
(i) dissolving Compound (I) in a suitable solvent(s) at room temperature;
(ii) stirring the mixture for a period of time;
(iii) slowly heating the mixture in step (ii) to a temperature to obtain a solution or slurry and holding the mixture at this temperature for a period of time;
(iv) slowly cooling the mixture obtained in step (iii);
(v) leaving the mixture from step (iii) to stand, with stirring, at room temperature until crystals are formed; and
(vi) isolating the crystals to obtain Compound (I), Form I with solvent(s).

In step (i), a suitable solvent is, for example, acetone, methanol, ethanol (e.g., denatured, 200 proof or 100% pure), acetonitrile, tetrahydrofuran, acetone/water, methanol/water, ethanol/water or tetrahydrofuran/heptane.

The resulting crystals of Form I may be recovered by any conventional methods known in the art.

In the final step (vi), the resulting solids obtained in step (v) may be collected and dried at high temperature using conventional collection and high-temperature drying techniques, for example, filtration and vacuum oven.

The process steps may of course be facilitated by conventional agitation techniques, e.g., stirring, and other conventional techniques as would be well understood for facilitation of the process.

Compound (I), Form II

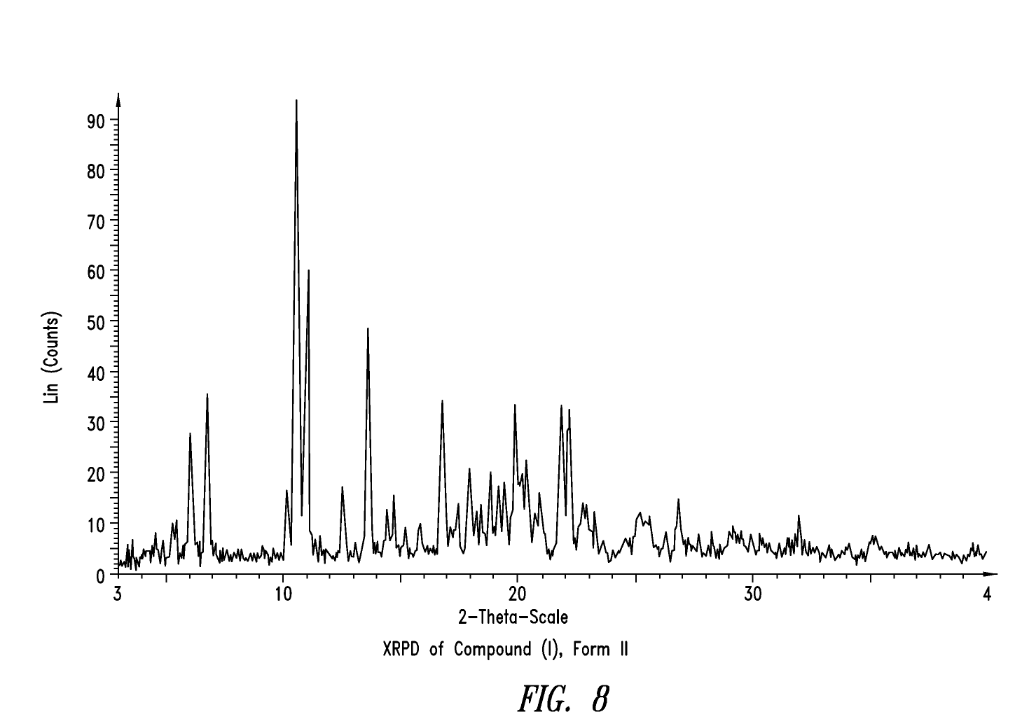

Form II is a solvate crystalline form. The XRPD pattern of Compound (I), Form II, is shown in FIG. 8. The characteristic peak positions and relative intensities for the XRPD pattern in FIG. 8 for Form II are shown in Table 4:

Compound (I), Type B

| Angle | |
|---|---|
| 2-Theta ° | Relative Intensity % |
| 6.0 | 30 |
| 6.7 | 39 |
| 10.5 | 100 |
| 10.9 | 54 |
| 12.5 | 18 |
| 13.5 | 55 |
| 16.7 | 34 |
| 17.8 | 27 |
| 19.8 | 36 |
| 21.8 | 37 |

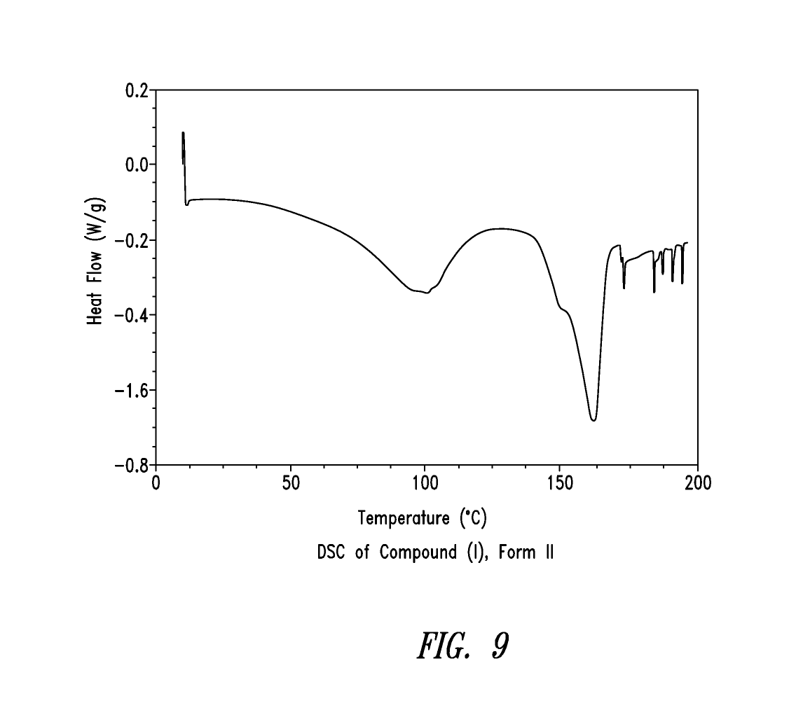

FIG. 9 shows the DSC thermal curve for Form II crystals obtained from a methyl-t-butyl ether/water solvent system where the DSC is performed at a heating rate of 10° C. per minute in a crimped cup.

An embodiment of the invention is directed to a crystaliine polymorph of Compound (I), Form II, having an X-ray powder diffraction pattern comprising peaks at 6.0, 6.7 and 13.5 degrees 2θ (±0.2 degrees 2θ) when measured using CuKα radiation.

Another embodiment is directed to a crystalline polymorph of Compound (I), Form II, having an XRPD pattern comprising peaks at 6.0, 6.7 and 13.5 degrees 2θ (±0.2 degrees 2θ) and further comprising peaks at 10.5, 10.9 and 16.7 degrees 2θ (±0.2 degrees 2θ) when measured using CuKα radiation.

Another embodiment is directed to a crystalline polymorph of Compound (I), Form II, having an XRPD pattern comprising peaks at 6.0, 6.7 and 13.5 degrees 2θ (±0.2 degrees 2θ) and further comprising peaks at 10.5, 10.9, 12.5, 16.7, 17.8, 19.8 and 21.8 degrees 2θ (±0.2 degrees 2θ) when measured using CuKα radiation.

Another embodiment is directed to a crystalline polymorph of Compound (I), Form II, having an XRPD pattern substantially the same as that shown in FIG. 8.

An embodiment of the invention is directed to a crystalline polymorph of a hydrochloride salt of Compound (I), Form II, exhibiting a DSC thermal curve substantially the same as that shown in FIG. 9.

Another embodiment is directed to a crystalline polymorph of a hydrochloride salt of Compound (I), Form II, having an XRPD pattern comprising peaks at 6.0, 6.7 and 13.5 degrees 2θ (±0.2 degrees 2θ) as described above and also exhibiting a DSC thermal curve substantially the same as that shown in FIG. 9.

Another embodiment is directed to a quantity of Compound (I) wherein at least 50%, preferably at least 75%, more preferably at least 95%, more preferably at least 99%, more preferably 100%, of said substance is present in crystalline form, for example, in the form of the Form II crystalline polymorph as characterized by any of the abovementioned XRPD-defined embodiments. The presence of such amounts of Form II in a quantity of Compound (I) is typically measurable using XRPD analysis of the compound.

An additional embodiment is directed to a pharmaceutical composition comprising Compound (I) and a pharmaceutically acceptable carrier or diluent, wherein at least 50%, preferably at least 75%, more preferably at least 95%, more preferably at least 99%, more preferably 100%, of the Compound (I) in the composition is present in crystalline form, for example, in the form of the Form II crystalline polymorph as defined above.

An additional embodiment is directed to a pharmaceutical composition comprising Compound (I) and a pharmaceutically acceptable carrier or diluent and further comprising at least one other antiviral agent, wherein at least 50%, preferably at least 75%, more preferably at least 95%, more preferably at least 99%, more preferably 100%, of the Compound (I) in the composition is present in crystalline form, for example, in the form of the Form II crystalline polymorph as defined above.

The present invention provides a process for the preparation of Form II which comprises crystallizing Compound (I) from a solution in solvents under conditions which yield Form II. The precise conditions under which Form II is formed may be empirically determined and it is only possible to give methods which have been found to be suitable in practice. As one of skill in the art will appreciate, in each of the following synthetic processes, the recited steps may (i) occur individually or one or more steps may combined into a singe step, (ii) occur in the order recited or in an alternative order and (iii) occur optionally.

It has been found that Compound (I), Form II may be prepared by a process comprising the following steps, which process is also an embodiment of the present invention:
(i) dissolving Compound (I) in a suitable solvent(s) at room temperature;
(ii) slowly heating the mixture in step (i) to a temperature to obtain a solution;
(iii) slowly cooling the solution obtained in step (ii), for example at a rate of 5° C./hour:
(iv) leaving the mixture from step (iii) to stand, with stirring, until crystals are formed; and
(v) isolating the precipitated crystals obtain Compound (I), Form II with solvent.

In step (i), a suitable solvent includes, for example, methyl-t-butyl ether, methyl-t-butyl ether/water or butyl acetate, preferably methyl-t-butyl ether.

The resulting crystals of Form II may be recovered by any conventional methods known in the art.

In the final step (v), the resulting solids obtained in step (iv) may be collected and dried at high temperature using conventional collection and high-temperature drying techniques, for example, filtration and vacuum oven.

The process steps may of course be facilitated by conventional agitation techniques, e.g., stirring, and other conventional techniques as would be well understood for facilitation the process.

Compound (I), Form III

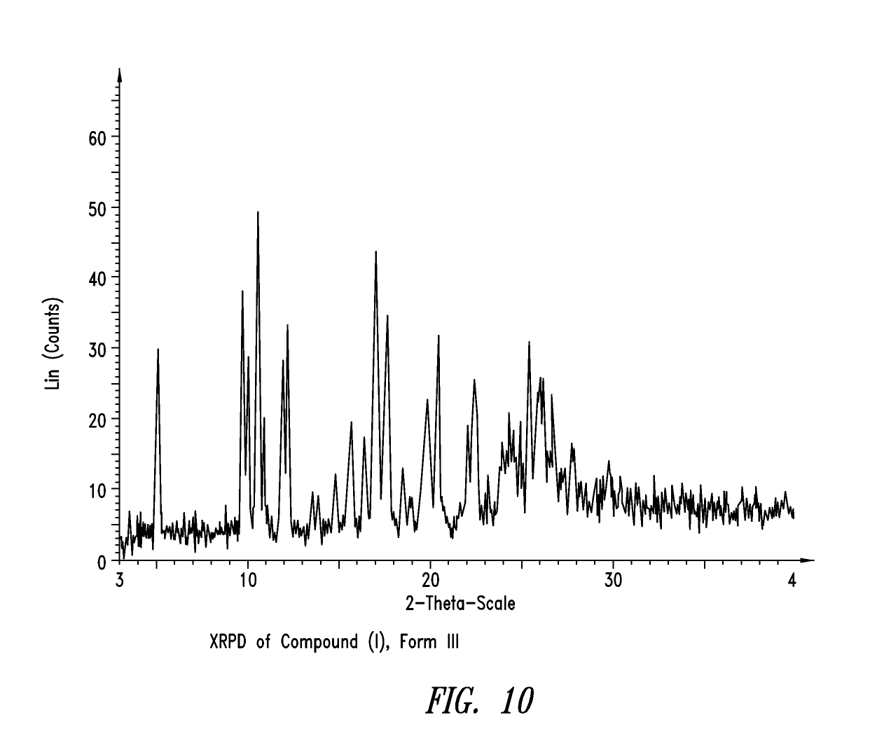

The XRPD pattern of Compound (I), Form III is shown in FIG. 10. Form III is a non-solvate crystalline form. The characteristic peak positions and relative intensities for the XRPD pattern in FIG. 10 for Form III are shown in Table 5:

Compound (I), Form III

| Angle | |
|---|---|
| 2-Theta ° | Relative Intensity % |
| 5.0 | 60 |
| 9.7 | 78 |
| 10.0 | 58 |
| 10.5 | 100 |
| 10.9 | 41 |
| 11.8 | 57 |
| 12.2 | 67 |
| 13.5 | 19 |
| 13.8 | 18 |
| 14.8 | 24 |
| 15.6 | 39 |
| 16.4 | 35 |
| 17.0 | 89 |
| 17.6 | 70 |
| 19.8 | 46 |

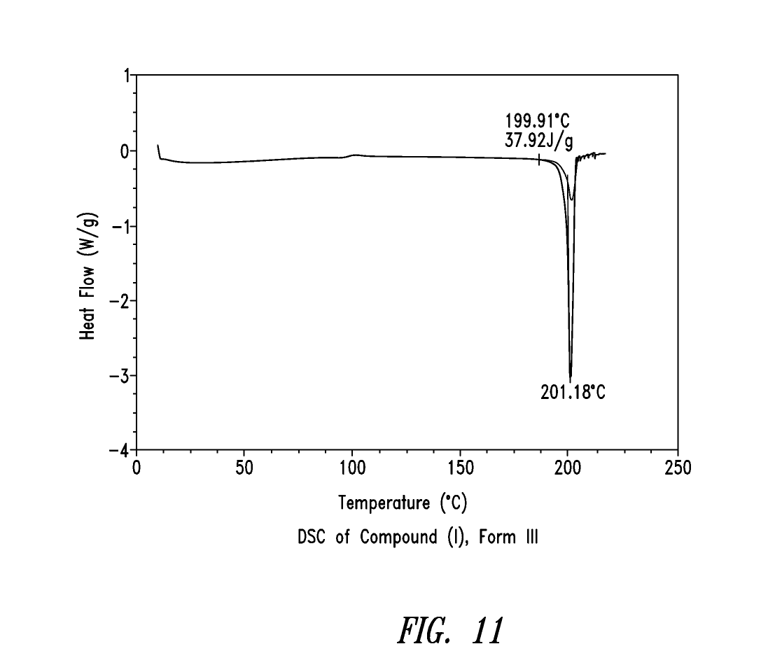

FIG. 11 shows the DSC thermal curve for Form III crystals where the DSC is performed at a heating rate of 10° C. per minute in a crimped cup.

An embodiment of the invention is directed to a crystalline polymorph of Compound (I), Form III, having an X-ray powder diffraction pattern comprising a peak at 5.0 and 16.4 degrees 2θ (±0.2 degrees 2θ) when measured using CuKα radiation.

Another embodiment is directed to a crystalline polymorph of Compound (I), Form III, having an XRPD pattern comprising a peak at 5.0 and 16.4 degrees 2θ (±0.2 degrees 2θ) and further comprising peaks at 9.7, 10.0, 10.5, 10.9, 11.8, 12.2, 13.5, 13.8, 14.8, 15.6, 17.0, 17.6 and 19.8 degrees 2θ (±0.2 degrees 2θ) when measured using CuKα radiation.

Another embodiment is directed to a crystalline polymorph of Compound (I), Form III, exhibiting an XRPD pattern substantially the same as that shown in FIG. 10.

An embodiment of the invention is directed to a crystalline polymorph of Compound (I). Form III, having a DSC thermal curve substantially the same as that shown in FIG. 11.

An embodiment of the invention is directed to a crystalline polymorph of Compound (I), Form III having an XRPD pattern comprising peaks at 5.0 and 16.4 degrees 2θ (±0.2 degrees 2θ) as described above and also exhibiting a DSC thermal curve substantially the same as that shown in FIG. 11.

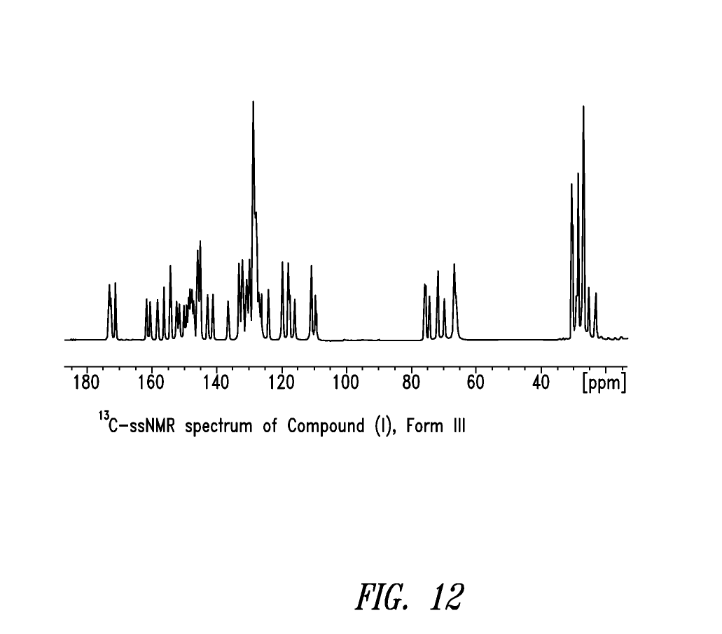

FIG. 12 is the ¹³C-solid-state Nuclear Magnetic Resonance (ssNMR) spectrum of crystalline polymorph of Compound (I), Form III.

An embodiment of the invention is directed to a crystalline polymorph of Compound (I). Form III, having a ¹³C-ssNMR spectrum having chemical shift peaks at 173.1, 172.6, 161.5, 160.4, 152.3, 151.4, 145.8, 141.1, 123.9, 119.6, 30.3, 26.8 and 25.1 ppm (each peak is ±0.2 ppm).

Another embodiment of the invention is directed to a crystalline polymorph of Compound (I), Form III, having a ¹³C-ssNMR spectrum having chemical shift peaks at 173.1, 172.6, 161.5, 160.4, 152.3, 151.4, 145.8, 141.1, 123.9, 119.6, 30.3, 26.8 and 25.1 ppm and further comprising chemical shift peaks at 171.1, 158.1, 156.2. 154.2, 150.0, 149.2, 148.5, 147.5, 147.0, 145.1 and 142.7 ppm (each peak is ±0.2 ppm).

Another embodiment of the invention is directed to a crystalline polymorph of Compound (I), Form III, having a ¹³C-ssNMR spectrum having chemical shift peaks at 173.1, 172.6, 161.5, 160.4, 152.3, 151.4, 145.8, 1411, 123.9, 119.6, 30.3, 26.8 and 25.1 ppm and further comprising chemical shift peaks at 171.1, 158.1, 156.2, 154.2, 150.0, 149.2, 148.5, 147.5, 147.0, 145.1, 142.7, 28.5 and 23.1 ppm (each peak is ±0.2 ppm).

Another embodiment of the invention is directed to a crystalline polymorph of Compound (I), Form III, having a ¹³C-ssNMR spectrum having chemical shift peaks at 173.1, 172.6, 161.5, 160.4, 152.3, 151.4, 145.8, 141.1, 123.9, 119.6, 30.3, 26.8 and 25.1 ppm and further comprising chemical shift peaks at 171.1, 158.1, 156.2, 154.2, 150.0, 149.2, 148.5, 147.5, 147.0, 145.1, 142.7, 136.4, 132.9, 131.9, 130.6, 129.8, 128.6, 127.7, 126.8, 126.1, 117.8, 117.4, 115.8, 110.7, 109.4, 75.8, 75.5, 74.2, 71.7, 69.8, 66.7, 28.5 and 23.1 ppm (each peak is ±0.2 ppm).

Another embodiment is directed to a crystalline polymorph of Compound (I), Form III, having an XRPD pattern comprising peaks at 5.0 and 16.4 degrees 2θ (±0.2 degrees 2θ) as described above or a ¹³C-ssNMR spectrum having chemical shift peaks at 173.1, 172.6, 161.5, 160.4, 152.3, 151.4, 145.8, 141.1, 123.9, 119.6, 30.3, 26.8 and 25.1 ppm (each peak is ±0.2 ppm).

Another embodiment is directed to a crystalline polymorph of Compound (I), Form III, having an XRPD pattern comprising peaks at 5.0 and 16.4 degrees 2θ (±0.2 degrees 2θ) as described above and also a ¹³C-ssNMR spectrum having chemical shift peaks at 173.1, 172.6, 161.5, 160.4, 152.3, 151.4, 145.8, 141.1, 123.9, 119.6, 30.3, 26.8 and 25.1 ppm (each peak is ±0.2 ppm).

Another embodiment is directed to a crystalline polymorph of Compound (I). Form III, exhibiting a ¹³C-ssNMR spectrum substantially the same as that shown in FIG. 12.

The present invention provides a process for the preparation of Form III which comprises crystallizing Compound (I) from a solution in solvents under conditions which yield Form III. The precise conditions under which Form III is formed may be empirically determined and it is only possible to give methods which have been found to be suitable in practice. As one of skill in the art will appreciate, in each of the following synthetic processes, the recited steps may (i) occur individually or one or more steps may combined into a singe step, (ii) occur in the order recited or in an alternative order and (iii) occur optionally.

It has been found that Compound (I), Form III may be prepared by a process comprising the following steps, which process is also an embodiment of the present invention:
(i) slurrying Compound (I), Form II in water:
(ii) slowly heating the mixture to a temperature to obtain a slurry, and leaving the mixture to stand, with stirring, at this temperature for a period of time;
(iii) slowly cooling the slurry obtained in step (ii), for example at a rate of 5° C./hour; and
(iv) isolating the crystals to obtain Compound (I), Form III.

In the final step (iv), the resulting solids obtained in step (iii) may be collected and dried at high temperature using conventional collection and high-temperature drying techniques, for example, filtration and vacuum oven.

The process steps may of course be facilitated by conventional agitation techniques, e.g., stirring, and other conventional techniques as would be well understood for facilitation the process.

Another embodiment is directed to a quantity of Compound (I) wherein at least 50%, preferably at least 75%, more preferably at least 95%, more preferably at least 99%, more preferably 100%, of said substance is present in crystalline form, for example, in the form of the Form III crystalline polymorph as characterized by any of the abovementioned XRPD-defined embodiments. The presence of such amounts of Form III in a quantity of Compound (I) is typically measurable using XRPD analysis of the compound.

An additional embodiment is directed to a pharmaceutical composition comprising Compound (I) and a pharmaceutically acceptable carrier or diluent, wherein at least 50%, preferably at least 75%, more preferably at least 95%, more preferably at least 99%, more preferably 100%, of the Compound (I) in the composition is present in crystalline form, for example, in the form of the Form III crystalline polymorph as defined above.

An additional embodiment is directed to a pharmaceutical composition comprising Compound (I) and a pharmaceutically acceptable carrier or diluent and further comprising at least one other antiviral agent, wherein at least 50%, preferably at least 75%, more preferably at least 95%, more preferably at least 99%, more preferably 100%, of the Compound (I) in the composition is present in crystalline form, for example, in the form of the Form III crystalline polymorph as defined above.

Pharmaceutical Compositions and Methods

The aforementioned crystalline forms of Compound (I), including Form I, Form II and Form III, the non-crystalline forms of the hydrochloride salt of Compound (I), the crystalline forms of the hydrochloride salt of Compound (I), including Type A and Type B, are useful as anti-HIV agents in view of the demonstrated inhibitory activity of Compound (I) against HIV integrase. These forms are therefore useful in treatment of HIV infection in a human and can be used for the preparation of a pharmaceutical composition for treating an HIV infection or alleviating one or more symptoms thereof in a patient. The appropriate dosage amounts and regimens for a particular patient can be determined by methods known in the art and by reference to the disclosure in WO 2007/131350 and WO 2009/062285. Generally, a therapeutically effective amount for the treatment of HIV infection in the human is administered. In one embodiment, about 50 mg to 1000 mg, more preferably from about 50 mg to about 400 mg, is administered per adult human per day in single or multiple doses.

Specific optimal dosage and treatment regimens for any particular patient will of course depend upon a variety of factors, including the age, body weight, general health status, sex, diet, time of administration, rate of excretion, drug combination, the severity and course of the infection, the patient's disposition to the infection and the judgment of the treating physician. In general, the compound is most desirably administered at a concentration level that will generally afford antivirally effective results without causing any harmful or deleterious side effects.

The crystalline forms of Compound (I) or the hydrochloride salt thereof at a selected dosage level is typically administered to the patient via a pharmaceutical composition. See, e.g., the description in WO 2007/131350 and WO 2009/062285 for the various types of compositions that may be employed in the present invention. The pharmaceutical composition may be administered orally, parenterally or via an implanted reservoir. The term parenteral as used herein includes subcutaneous, intracutaneous, intravenous, intramuscular, intra-articular, intrasynovial, intrasternal, intrathecal, and intralesional injection or infusion techniques. Oral administration or administration by injection are preferred.

The pharmaceutical compositions of this invention may contain any conventional non-toxic pharmaceutically-acceptable carriers, diluents, adjuvants, excipients or vehicles. In some cases, the pH of the formulation may be adjusted with pharmaceutically acceptable acids, bases or buffers to enhance the stability of the formulated compound or its delivery form.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents.

The pharmaceutical compositions may also be in the form of separate oral pharmaceutical compositions comprising crystalline Compound (I), Form I, Form II, or Form III, non-crystalline hydrochloride salt of Compound (I), or a crystalline hydrochloride salt of Compound (I), Type A or Type B, and at least one pharmaceutically acceptable carrier or diluent. The pharmaceutical compositions may also be in the form of separate oral pharmaceutical compositions comprising crystalline Compound (I). Form I, Form II, or Form III, non-crystalline hydrochloride salt of Compound (I), or a crystalline hydrochloride salt of Compound (I), Type A or Type B, and one or more further antiviral agent. The oral pharmaceutical compositions may be orally administered in any orally acceptable dosage form including, but not limited to, tablets, capsules (e.g., hard or soft gelatin capsules), including liquid-filled capsules, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. Examples of soft gelatin capsules that can be used include those disclosed in U.S. Pat. No. 5,985,321. When aqueous suspensions are administered orally, the active ingredient is combined with emulsifying and suspending agents. If desired, certain sweetening and/or flavoring and/or coloring agents may be added.

Other suitable vehicles or carriers for the above noted formulations and compositions can be found in standard pharmaceutical texts, e.g., in “Remington's Pharmaceutical Sciences”, 19^{th} ed., Mack Publishing Company, Easton, Pa., 1995.

Certainly, when the crystalline hydrochloride salt of Compound (I), Type A or Type B is formulated in a liquid vehicle, for example, as a liquid solution or suspension for oral administration or by injection, including for example in liquid-filled capsules, the crystalline hydrochloride salt of Compound (I), Type A and Type B lose their crystalline nature. Nevertheless, the final liquid-based pharmaceutical composition contains the novel hydrochloride salt of Compound (I) and it is therefore to be considered a separate embodiment embraced by the present invention. It was only by discovering a method for preparing the hydrochloride salt in a stable crystalline form that the present inventors enabled efficient pharmaceutical processing and pharmaceutical formulation manufacture using the hydrochloride salt form. Therefore, the final pharmaceutical formulation containing the hydrochloride salt form which was thereby enabled by this discovery is considered another aspect and embodiment of the present invention.

Methods of Characterization

X-Ray Powder Diffraction

X-ray powder diffraction analyses were conducted on a Bruker AXS X-Ray Powder Diffractometer Model D8 Advance, available from Bruker AXS, Inc. of Madison, Wis., using CuKα radiation (1.54 Å). The tube power was set to 40 kV and 40 mA. Step scans were run from 2 to 35° 2θ, at 0.05° per step, 4 sec per step. A reference quartz standard was used to check instrument alignment. Samples were prepared for analysis by filling a zero background quartz holder.

DSC Analysis

The DSC analysis was conducted on a TA instruments DSC Q 1000. The differential scanning calorimetry curve was obtained on a sample heated at 10° C. in a crimped cup under a nitrogen flow.

Solid-State NMR (ssNMR)

ssNMR data was acquired on a Bruker Avance III NMR spectrometer (Bruker Biospin, Inc., Billerica, Mass.) at 9.4 T (¹H=400.46 MHz. ¹³C=100.70 MHz). Samples were packed in 4 mm O.D. zirconia rotors with Kel-F® drive tips. A Bruker model 4BL CP BB WVT probe was used for data acquisition and sample spinning about the magic-angle (54.74°). Sample spectrum acquisition used a spinning rate of 14 kHz. A standard cross-polarization pulse sequence was used with a ramped Hartman-Hahn match pulse on the proton channel at ambient temperature and pressure. The pulse sequence used a 5 millisecond contact pulse and a 3 second recycle delay. Two-pulse phase modulated (tppm) decoupling was also employed in the pulse sequence. No exponential line broadening was used prior to Fourier transformation of the free incution decay. Chemical shifts were referenced using the secondary standard of adamantane, with the upfield resonance being set to 29.5 ppm. The magic-angle was set using the ⁷⁹Br signal from KBr powder at a spinning rate of 5 kHz.

In order that this invention to be more fully understood, the following examples are set forth. These examples are for the purpose of illustrating embodiments of this invention, and are not to be construed as limiting the scope of the invention in any way. The reactants used in the examples below may be obtained either as described herein, or if not described herein, are themselves either commercially available or may be prepared from commercially available materials by methods known in the art. Certain starting materials, for example, may be obtained by methods described in the International Patent Applications WO 2007/131350 and WO 2009/062285.

Unless otherwise specified, solvents, temperatures, pressures, and other reaction conditions may be readily selected by one of ordinary skill in the art. Typically, reaction progress may be monitored by High Pressure Liquid Chromatography (HPLC), if desired, and intermediates and products may be purified by chromatography on silica gel and/or by recrystallization.

### EXAMPLES

Abbreviations or symbols used herein include:

Ac: acetyl; AcOH: acetic acid; Ac₂O: acetic anhydride; Bu: butyl; DMAc: N,N-Dimethylacetamide; ee: enantiomeric excess: Eq: equivalent; Et: ethyl; EtOAc: ethyl acetate; EtOH: ethanol; GC: gas chromatography; HPLC: high performance liquid chromatography; IPA: isopropyl alcohol; ⁱPr or i-Pr: 1-methylethyl (iso-propyl); KF: Karl Fischer; LOD: limit of detection; Me: methyl: MeCN: acetonitrile; MeOH: methanol; MS: mass spectrometry (ES: electrospray); MTBE; methyl-t-butyl ether, BuLi: n-butyl lithium; NMR: nuclear magnetic resonance spectroscopy: Pr propyl; tert-butyl or t-butyl: 1,1-dimethylethyl; TFA: trifluoroacetic acid and THF: tetrahydrofuran.

### Example 1

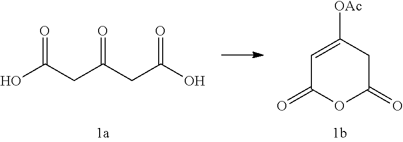 [SMILES: O=C(O)CC(=O)CC(=O)O ; CC(=O)OC1=CC(=O)OC(=O)C1]

1a (600 g, 4.1 mol) was charged into a dry reactor under nitrogen followed by addition of Ac₂O (1257.5 g, 12.3 mol, 3 eq.). The resulting mixture was heated at 40° C. at least for 2 hours. The batch was then cooled to 30° C. over 30 minutes. A suspension of 1b in toluene was added to seed the batch if no solid was observed. After toluene (600 mL) was added over 30 minutes, the batch was cooled to −5˜−10° C. and was held at this temperature for at least 30 minutes. The solid was collected by filtration under nitrogen and rinsed with heptanes (1200 mL). After being dried under vacuum at room temperature, the solid was stored under nitrogen at least below 20° C. The product 1b was obtained with 77% yield. ¹H NMR (500 MHz, CDCl₃): δ=6.36 (s, 1H), 3.68 (s, 2H), 2.30 (s, 3H).

### Example 2

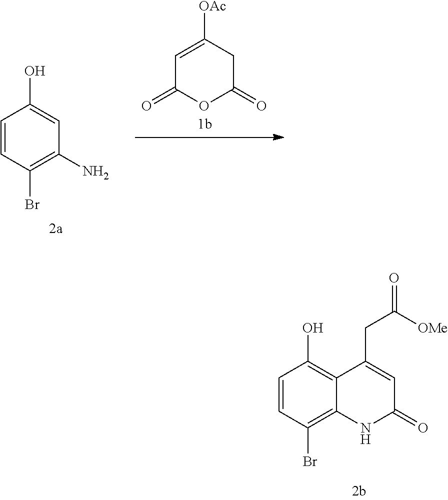 [SMILES: Nc1cc(O)ccc1Br ; CC(=O)OC1=CC(=O)OC(=O)C1 ; COC(=O)Cc1cc(=O)[nH]c2c(Br)ccc(O)c12]

2a (100 g, 531 mmol) and 1b (95 g, 558 mmol) were charged into a clean and dry reactor under nitrogen followed by addition of fluorobenzene (1000 mL). After being heated at 35-37° C. for 4 hours, the batch was cooled to 23° C. Concentrated H₂SO₄ (260.82 g, 2659.3 mmol, 5 eq.) was added while maintaining the batch temperature below 35° C. The batch was first heated at 30-35° C. for 30 minutes and then at 40-45° C. for 2 hours. 4-Methyl morpholine (215.19 g, 2127 mmol, 4 eq.) was added to the batch while maintaining the temperature below 50° C. Then the batch was agitated for 30 minutes at 40-50° C. MeOH (100 mL) was then added while maintaining the temperature below 55° C. After the batch was held at 50-55° C. for 2 hours, another portion of MeOH (100 mL) was added. The batch was agitated for another 2 hours at 50-55° C. After fluorobenzene was distilled to a minimum amount, water (1000 mL) was added. Further distillation was performed to remove any remaining fluorobenzene. After the batch was cooled to 30° C., the solid was collected by filtration with cloth and rinsed with water (400 mL) and heptane (200 mL). The solid was dried under vacuum below 50° C. to reach KF<0.1%. Typically, the product 2b was obtained in 90% yield with 98 wt %. ¹H NMR (500 MHz, DMSO-ds): δ=10.83 (s, 1H), 9.85 (s, bs, 1H), 7.6 (d, 1H, J=8.7 Hz), 6.55 (d, 1H, J=8.7 Hz), 6.40 (s, 1H), 4.00 (s, 2H), 3.61 (s, 3H).

### Example 3

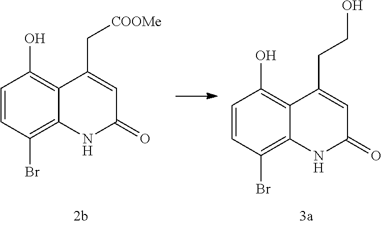 [SMILES: COC(=O)Cc1cc(=O)[nH]c2c(Br)ccc(O)c12 ; O=c1cc(CCO)c2c(O)ccc(Br)c2[nH]1]

2b (20 g, 64 mmol) was charged into a clean and dry reactor followed by addition of THF (140 mL). After the resulting mixture was cooled to 0° C., Vitride® (Red-Al, 47.84 g, 65 wt %, 154 mmol) in toluene was added while maintaining an internal temperature at 0-5° C. After the batch was agitated at 5-10° C. for 4 hours, IPA (9.24 g, 153.8 mmol) was added while maintaining the temperature below 10° C. Then the batch was agitated at least for 30 minutes below 25° C. A solution of HCl in IPA (84.73 g, 5.5 M, 512 mmol) was added into the reactor while maintaining the temperature below 40° C. After about 160 mL of the solvent was distilled under vacuum below 40° C., the batch was cooled to 20-25° C. and then aqueous 6M HCl (60 mL) was added while maintaining the temperature below 40° C. The batch was cooled to 25° C. and agitated for at least 30 minutes. The solid was collected by filtration, washed with 40 mL of IPA and water (1V/1V), 40 mL of water and 40 mL of heptanes. The solid was dried below 60° C. in a vacuum oven to reach KF<0.5%. Typically, the product 3a was obtained in 90-95% yield with 95 wt %. ¹H NMR (400 MHz, DMSO-d₆): δ=10.7 (s, 1H), 9.68 (s, 1H), 7.59 (d, 1H, J=8.7 Hz), 6.64 (1H, J=8.7 Hz), 6.27 (s, 1H), 4.62 (bs, 1H), 3.69 (t, 2H, J=6.3 Hz), 3.21 (t, 2H, J=6.3 Hz).

### Example 4

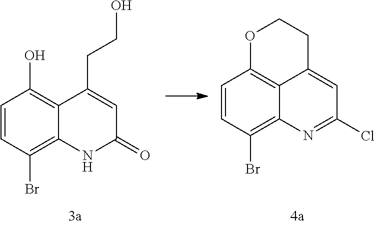 [SMILES: O=c1cc(CCO)c2c(O)ccc(Br)c2[nH]1 ; Clc1cc2c3c(ccc(Br)c3n1)OCC2]

3a (50 g, 174.756 mmol) and acetonitrile (200 mL) were charged into a dry and clean reactor. After the resulting mixture was heated to 65° C., POCl₃ (107.18 g, 699 mmol, 4 eq.) was added while maintaining the internal temperature below 75° C. The batch was then heated at 70-75° C. for 5-6 h. The batch was cooled to 20° C. Water (400 mL) was added at least over 30 minutes while maintaining the internal temperature below 50° C. After the batch was cooled to 20-25° C. over 30 minutes, the solid was collected by filtration and washed with water (100 mL). The wet cake was charged back into the reactor followed by addition of 1M NaOH (150 mL). After the batch was agitated at least for 30 minutes at 25-35° C., verify that the pH was greater than 12. Otherwise, more 6M NaOH was needed to adjust the pH>12. After the batch was agitated for 30 minutes at 25-35° C., the solid was collected by filtration, washed with water (200 mL) and heptanes (200 mL). The solid was dried in a vacuum oven below 50° C. to reach KF<2%. Typically, the product 4a was obtained at about 75-80% yield. ¹H NMR (400 MHz, CDCl₃): δ=7.90 (d, 1H, J=8.4 Hz), 7.16 (s, 1H), 6.89 (d, 1H, J=8.4 Hz), 4.44 (t, 2H, J=5.9 Hz), 3.23 (t, 2H, J=5.9 Hz). ¹³C NMR (100 MHz, CDCl₃): δ=152.9, 151.9, 144.9, 144.1, 134.6, 119.1, 117.0, 113.3, 111.9, 65.6, 28.3.

### Example 5

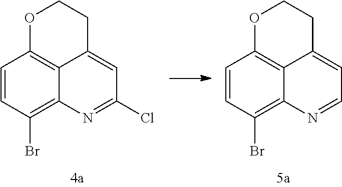 [SMILES: Clc1cc2c3c(ccc(Br)c3n1)OCC2 ; Brc1ccc2c3c(ccnc13)CCO2]

Zn powder (54 g, 825 mmol, 2.5 eq.) and TFA (100 mL) were charged into a dry and clean reactor. The resulting mixture was heated to 60-65° C. A suspension of 4a (100 g, 330 mmol) in 150 mL of TFA was added to the reactor while maintaining the temperature below 70° C. The charge line was rinsed with TFA (50 mL) into the reactor. After 1 hour at 65±5° C., the batch was cooled to 25-30° C. Zn powder was filtered off by passing the batch through a Celite pad and washing with methanol (200 mL). About 400 mL of solvent was distilled off under vacuum. After the batch was cooled to 20-25° C., 20% NaOAc (ca. 300 mL) was added at least over 30 minutes to reach pH 5-6. The solid was collected by filtration, washed with water (200 mL) and heptane (200 mL), and dried under vacuum below 45° C. to reach KF≦2%. The solid was charged into a dry reactor followed by addition of loose carbon (10 wt %) and toluene (1000 mL). The batch was heated at least for 30 minutes at 45-50° C. The carbon was filtered off above 35° C. and rinsed with toluene (200 mL). The filtrate was charged into a clean and dry reactor. After about 1000 mL of toluene was distilled off under vacuum below 50° C., 1000 mL of heptane was added over 30 minutes at 40-50° C. Then the batch was cooled to 0±5° C. over 30 minutes. After 30 minutes, the solid was collected and rinsed with 200 mL of heptane. The solid was dried under vacuum below 45° C. to reach KF≦500 ppm. Typically, the product 5a was obtained in about 90-95% yield. ¹H NMR (400 MHz, CDCl₃): δ=8.93 (m, 1H), 7.91 (dd, 1H, J=1.5, 8 Hz), 7.17 (m 1H), 6.90 (dd, 1H, J=1.6, 8.0 Hz), 4.46-4.43 (m, 2H), 3.28-3.23 (m, 2H). ¹³C NMR (100 MHz, CDCl₃): δ=152.8, 151.2, 145.1, 141.0, 133.3, 118.5, 118.2, 114.5, 111.1, 65.8, 28.4.

### Example 6

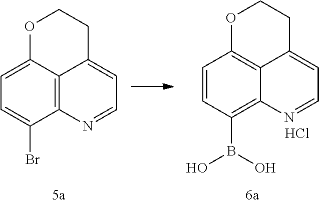 [SMILES: Brc1ccc2c3c(ccnc13)CCO2 ; OB(O)c1ccc2c3c(ccnc13)CCO2 ; Cl]

5a (1.04 kg, 4.16 mol) and toluene (8 L) were charged into the reactor. The batch was agitated and cooled to −50 to −55° C. BuLi solution (2.5 M in hexanes, 1.69 L, 4.23 mol) was charged slowly while maintaining the internal temperature between −45 to −50° C. The batch was agitated at −45° C. for 1 hour after addition. A solution of triisopropyl borate (0.85 kg, 4.5 mol) in MTBE (1.48 kg) was charged. The batch was warmed to 10° C. over 30 minutes. A solution of 5 N HCl in IPA (1.54 L) was charged slowly at 10° C., and the batch was warmed to 20° C. and stirred for 30 minutes. It was seeded with 6a crystal (10 g). A solution of aqueous concentrated HCl (0.16 L) in IPA (0.16 L) was charged slowly at 20° C. in three portions at 20 minute intervals, and the batch was agitated for 1 hour at 20° C. The solid was collected by filtration, rinsed with MTBE (1 kg), and dried to provide 6a (943 g, 88.7% purity, 80% yield). ¹H NMR (400 MHz, 020): δ 8.84 (d, 1H, J=4 Hz), 8.10 (m, 1H), 7.68 (d, 1H, J=6 Hz), 7.09 (m, 1H), 4.52 (m, 2H), 3.47 (m, 2H).

### Example 7

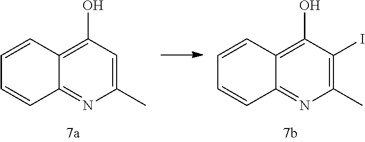

Iodine stock solution was prepared by mixing iodine (57.4 g, 0.23 mol) and sodium iodide (73.4 g, 0.49 mol) in water (270 mL). Sodium hydroxide (28.6 g, 0.715 mol) was charged into 220 mL of water. 4-Hydroxy-2 methylquinoline 7a (30 g, 0.19 mol) was charged, followed by acetonitrile (250 mL). The mixture was cooled to 10° C. with agitation. The above iodine stock solution was charged slowly over 30 minutes. The reaction was quenched by addition of sodium bisulfite (6.0 g) in water (60 mL). Acetic acid (23 mL) was charged over a period of 1 hour to adjust the pH of the reaction mixture between 6 and 7. The product was collected by filtration, washed with water and acetonitrile, and dried to give 7b (53 g, 98%). MS 286 [M+1].

### Example 8

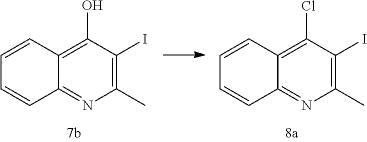

4-Hydroxy-3-iodo-2-methylquinoline 7b (25 g, 0.09 mol) was charged to a 1-L reactor. Ethyl acetate (250 mL) was charged, followed by triethylamine (2.45 mL, 0.02 mol) and phosphorus oxychloride (12 mL, 0.13 mol). The reaction mixture was heated to reflux until complete conversion (˜1 hour), then the mixture was cooled to 22° C. A solution of sodium carbonate (31.6 g, 0.3 mol) in water (500 mL) was charged. The mixture was stirred for 20 minutes. The aqueous layer was extracted with ethyl acetate (120 mL). The organic layers were combined and concentrated under vacuum to dryness. Acetone (50 mL) was charged. The solution was heated to 60° C. Water (100 mL) was charged, and the mixture was cooled to 22° C. The product was collected by filtration and dried to give 8a (25 g, 97.3% pure, 91.4% yield). MS 304 [M+1].

(Note: 8a is a known compound with CAS #1033931-93-9. See references: (a) J. Org Chem. 2008, 73, 4644-4649. (b) Molecules 2010, 15, 3171-3178. (c) Indian J. Chem. Sec B: Org. Chem. Including Med Chem. 2009, 488(5), 692-696.)

### Example 9

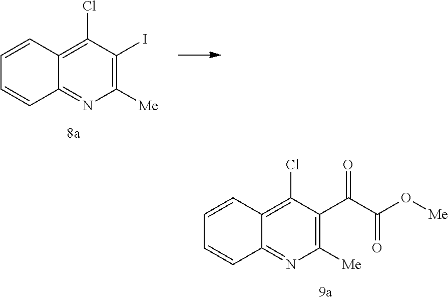 [SMILES: Cc1nc2ccccc2c(Cl)c1I ; COC(=O)C(=O)c1c(C)nc2ccccc2c1Cl]

8a (100 g, 0.33 mol) was charged to the reactor, followed by copper (I) bromide dimethyl sulfide complex (3.4 g, 0.017 mol) and dry THF (450 mL). The batch was cooled to −15 to −12° C. i-PrMgCl (2.0 M in THF, 173 mL, 0.346 mol) was charged into the reactor at the rate which maintains the batch temperature <−10° C. In a 2nd reactor, methyl chlorooxoacetate (33 mL, 0.36 mol) and dry THF (150 mL) was charged. The solution was cooled to −15 to −10° C. The content of the 1st reactor (Grignard/cuprate) was charged into the 2nd reactor at the rate which maintained the batch temperature <−10° C. The batch was agitated for 30 minutes at −10° C. Aqueous ammonium chloride solution (10%, 300 mL) was charged. The batch was agitated at 20-25° C. for 20 minutes and allowed to settle for 20 minutes. The aqueous layer was separated. Aqueous ammonium chloride solution (10%, 90 mL) and sodium carbonate solution (10%, 135 mL) were charged to the reactor. The batch was agitated at 20-25° C. for 20 minutes and allowed to settle for 20 minutes. The aqueous layer was separated. Brine (10%, 240 mL) was charged to the reactor. The batch was agitated at 20-25° C. for 20 minutes. The aqueous layer was separated. The batch was concentrated under vacuum to ˜¼ of the volume (about 80 mL left). 2-Propanol was charged (300 mL). The batch was concentrated under vacuum to ˜⅓ of the volume (about 140 mL left), and heated to 50° C. Water (70 mL) was charged. The batch was cooled to 20-25° C., stirred for 2 hours, cooled to ˜10° C. and stirred for another 2 hours. The solid was collected by filtration, washed with cold 2-propanol and water to provide 58.9 g of 9a obtained after drying (67.8% yield). ¹H NMR (400 MHz, CDCl₃): δ 8.08 (d, 1H, J=12 Hz), 7.97 (d, 1H J=12 Hz), 7.13 (t, 1H, J=8 Hz), 7.55 (t, 1H, J=8 Hz), 3.92 (s, 3H), 2.63 (s, 3H). ¹³C NMR (100 MHz, CDCl₃): δ 186.6, 161.1, 155.3, 148.2, 140.9, 132.0, 129.0, 128.8, 127.8, 123.8, 123.7, 53.7, 23.6.

### Example 10

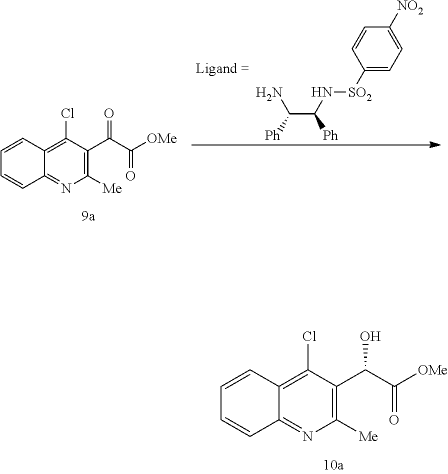 [SMILES: COC(=O)C(=O)c1c(C)nc2ccccc2c1Cl ; N[C@@H](c1ccccc1)[C@@H](NS(=O)(=O)c1ccc([N+](=O)[O-])cc1)c1ccccc1 ; COC(=O)[C@@H](O)c1c(C)nc2ccccc2c1Cl]

Catalyst Preparation:

To a suitable sized, clean and dry reactor was charged dichloro(pentamethylcyclopentadienyl)rhodium(III) dimer (800 ppm relative to 9a, 188.5 mg) and the ligand (2000 ppm relative to 9a, 306.1 mg). The system was purged with nitrogen and then 3 mL of acetonitrile and 0.3 mL of triethylamine was charged to the system. The resulting solution was agitated at RT for not less than 45 minutes and not more than 6 hours.

Reaction:

To a suitable sized, clean and dry reactor was charged 9a (1.00 equiv, 100.0 g (99.5 wt %), 377.4 mmol). The reaction was purged with nitrogen. To the reactor was charged acetonitrile (ACS grade, 4 L/Kg of 9a, 400 mL) and triethylamine (2.50 equiv, 132.8 mL, 943 mmol). Agitation was initiated. The 9a solution was cooled to Tᵢₙₜ=−5 to 0° C. and then formic acid (3.00 equiv, 45.2 mL, 1132 mmol) was charged to the solution at a rate to maintain Tᵢₙₜ not more than 20° C. The batch temperature was then adjusted to Tᵢₙₜ=−5 to −0° C. Nitrogen was bubbled through the batch through a porous gas dispersion unit (Wilmad-LabGlass No. LG-8680-110, VWR catalog number 14202-962) until a fine stream of bubbles was obtained. To the stirring solution at Tᵢₙₜ=−5 to 0° C. was charged the prepared catalyst solution from the catalyst preparation above. The solution was agitated at Tᵢₙₜ=−5 to 0° C. with the bubbling of nitrogen through the batch until HPLC analysis of the batch indicated no less than 98 A % conversion (as recorded at 220 nm, 10-14 h). To the reactor was charged isopropylacetate (6.7 L/Kg of 9a, 670 mL). The batch temperature was adjusted to Tᵢₙₜ=18 to 23° C. To the solution was charged water (10 L/Kg of 9a, 1000 mL) and the batch was agitated at Tᵢₙₜ=18 to 23° C. for no less than 20 minutes. The agitation was decreased and or stopped and the layers were allowed to separate. The lighter colored aqueous layer was cut. To the solution was charged water (7.5 L/Kg of 9a, 750 mL) and the batch was agitated at Tᵢₙₜ=18 to 23° C. for no less than 20 minutes. The agitation was decreased and or stopped and the layers were allowed to separate. The lighter colored aqueous layer was cut. The batch was then reduced to 300 mL (3 L/Kg of 9a) via distillation while maintaining Tₑₓₜ no more than 65° C. The batch was cooled to Tᵢₙₜ=35 to 45° C. and the batch was seeded (10 mg). To the batch at Tᵢₙₜ=35 to 45° C. charged heptane (16.7 L/Kg of 9a, 1670 mL) over no less than 1.5 hours. Adjusted the batch temperature to Tᵢₙₜ=−2 to 3° C. over no less than 1 hour, and agitated the batch at Tᵢₙₜ=−2 to 3° C. for no less than 1 hour. Collected the solids by filtration. Used the filtrate to rinse the reactor (Filtrate is cooled to Tᵢₙₜ=−2 to 3° C. before filtration) and the solids were suction dried for no less than 2 hours. The solids were dried until the LOD was no more than 4% to obtain 82.7 g of 10a (99.6-100 wt %, 98.5% ee, 82.5% yield). ¹H-NMR (CDCl₃, 400 MHz) δ: 8.20 (d, J=8.4 Hz, 1H), 8.01 (d. J=8.4 Hz, 1H), 7.73 (t, J=7.4 Hz, 1H), 7.59 (t, J=7.7 Hz, 1H), 6.03 (s, 1H), 3.93 (s, 1H), 3.79 (s, 3H), 2.77 (s, 3H). ¹³C-NMR (CDCl₃, 100 MHz) δ: 173.5, 158.3, 147.5, 142.9, 130.7, 128.8, 127.7, 127.1, 125.1, 124.6, 69.2, 53.4, 24.0.

### Example 11

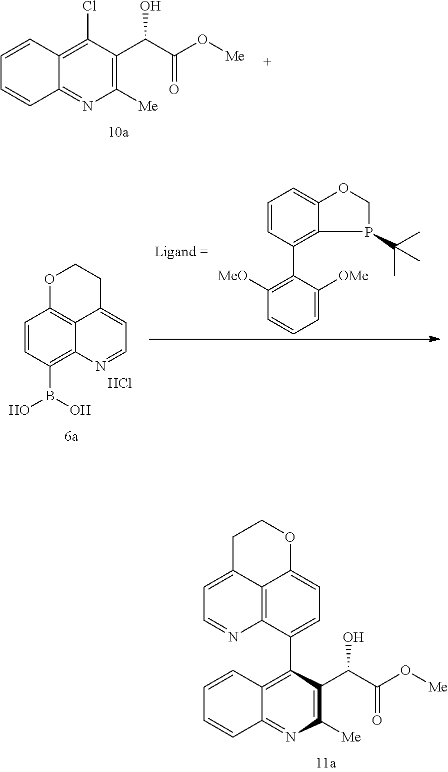

10a (2.45 kg, 96.8% purity, 8.9 mol), 6a (2.5 kg, 88.7% purity, 8.82 mol), tris(dibenzylideneacetone)dipalladium(0) (Pd₂ dba, 40 g, 0.044 mol), (S)-3-tert-butyl-4-(2,6-dimethoxyphenyl)-2,3-dihydrobenzo[d][1,3]oxaphosphole (32 g, 0.011 mol), sodium carbonate (1.12 kg, 10.58 mol), 1-pentanol (16.69 L), and water (8.35 L) were charged to the reactor. The mixture was de-gassed by sparging with argon for 10-15 minutes, was heated to 60-63° C., and was agitated until HPLC analysis of the reaction shows <1 A % (220 nm) of the 6a relative to the combined two atropisomer products (˜15 hours). The batch was cooled to 18-23° C. Water (5 L) and heptane (21 L) were charged. The slurry was agitated for 3-5 hours. The solids were collected by filtration, washed with water (4 L) and heptane/toluene mixed solvent (2.5 L toluene/5 L heptane), and dried. The solids were dissolved in methanol (25 L) and the resulting solution was heated to 50° C. and circulated through a CUNO carbon stack filter. The solution was distilled under vacuum to ˜5 L. Toluene (12 L) was charged. The mixture was distilled under vacuum to ˜5 L and cooled to 22° C. Heptane (13 L) was charged to the contents over 1 hour and the resulting slurry was agitated at 20-25° C. for 3-4 hours. The solids were collected by filtration and washed with heptanes to provide 2.58 kg of 11a obtained after drying (73% yield). ¹H NMR (400 MHz, CDCl₃): δ 8.63 (d, 1H, J=8 Hz), 8.03 (d, 1H, J=12 Hz), 7.56 (t, 1H, J=8 Hz), 7.41 (d, 1H, J=8 Hz), 7.19 (t, 1H, J=8 Hz), 7.09 (m, 2H), 7.04 (d, 1H, J=8 Hz), 5.38 (d, 1H, J=8 Hz), 5.14 (d, 1H, J=8 Hz), 4.50 (t, 2H, J=4 Hz), 3.40 (s, 3H), 3.25 (t, 2H, J=4 Hz), 2.91 (s, 3H). ¹³C NMR (100 MHz, CDCl₃): δ 173.6, 158.2, 154.0, 150.9, 147.3, 147.2, 145.7, 141.3, 132.9, 123.0, 129.4, 128.6, 127.8, 126.7, 126.4, 125.8, 118.1, 117.3, 109.9, 70.3, 65.8, 52.3, 28.5, 24.0.

### Example 12

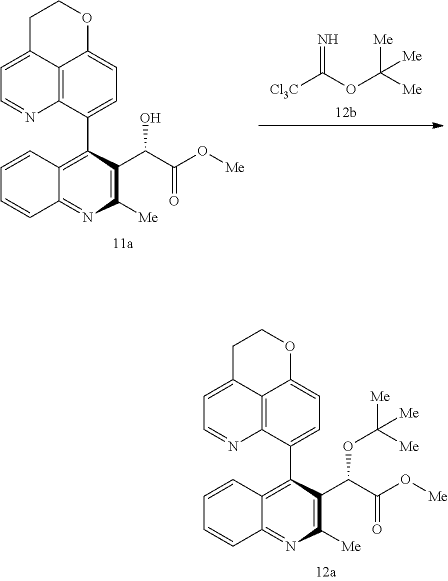

To a suitable clean and dry reactor under a nitrogen atmosphere was charged 11a (5.47 Kg, 93.4 wt %, 1.00 equiv, 12.8 mol) and fluorobenzene (10 vols, 51.1 kg) following by trifluoromethanesulfonimide (4 mol %, 143 g, 0.51 mol) as a 0.5 M solution in DCM (1.0 Kg). The batch temperature was adjusted to 35-41° C. and agitated to form a fine slurry. To the mixture was slowly charged t-butyl-2,2,2-trichloroacetimidate 12b as a 50 wt % solution (26.0 Kg of t-butyl-2,2,2-trichloroacetimidate (119.0 mol, 9.3 equiv), the reagent was −48-51 wt % with the remainder 52-49 wt % of the solution being ˜1.8:1 wt:wt heptane: fluorobenzene) over no less than 4 hours at Tᵢₙₜ=35-41° C. The batch was agitated at Tᵢₙₜ=35-41° C. until HPLC conversion (308 nm) was >96 A %, then cooled to Tᵢₙₜ=20-25° C. and then triethylamine (0.14 equiv, 181 g, 1.79 mol) was charged followed by heptane (12.9 Kg) over no less than 30 minutes. The batch was agitated at Tᵢₙₜ=20-25° C. for no less than 1 hour. The solids were collected by filtration. The reactor was rinsed with the filtrate to collect all solids. The collected solids in the filter were rinsed with heptane (11.7 Kg). The solids were charged into the reactor along with 54.1 Kg of DMAc and the batch temperature adjusted to Tᵢₙₜ=70-75° C. Water (11.2 Kg) was charged over no less than 30 minutes while the batch temperature was maintained at Tᵢₙₜ=65-75° C. 12a seed crystals (34 g) in water (680 g) was charged to the batch at Tᵢₙₜ=65-75° C. Additional water (46.0 Kg) was charged over no less than 2 hours while maintaining the batch temperature at Tᵢₙₜ=65-75° C. The batch temperature was adjusted to Tᵢₙₜ=18-25=C over no less than 2 hours and agitated for no less than 1 hour. The solids were collected by filtration and the filtrate used to rinse the reactor. The solids were washed with water (30 Kg) and dried under vacuum at no more than 45° C. until the LOD<4% to obtain 12a (5.275 Kg, 99.9 A % at 220 nm, 99.9 wt % via HPLC wt % assay, 90.5% yield). ¹H-NMR (CDCl₃, 400 MHz) δ: 8.66-8.65 (m, 1H), 8.05 (d, J=8.3 Hz, 1H), 7.59 (t, J=7.3 Hz, 1H), 7.45 (d, J=7.8 Hz, 1H), 7.21 (t, J=7.6 Hz, 1H), 7.13-7.08 (m, 3H), 5.05 (s, 1H), 4.63-4.52 (m, 2H), 3.49 (s, 3H), 3.41-3.27 (m, 2H), 3.00 (s, 3H), 0.97 (s, 9H). ¹³C-NMR (CDCl₃, 100 MHz) δ: 172.1, 159.5, 153.5, 150.2, 147.4, 146.9, 145.4, 140.2, 131.1, 130.1, 128.9, 128.6, 128.0, 127.3, 126.7, 125.4, 117.7, 117.2, 109.4, 76.1, 71.6, 65.8, 51.9, 28.6, 28.0, 25.4.

### Example 13

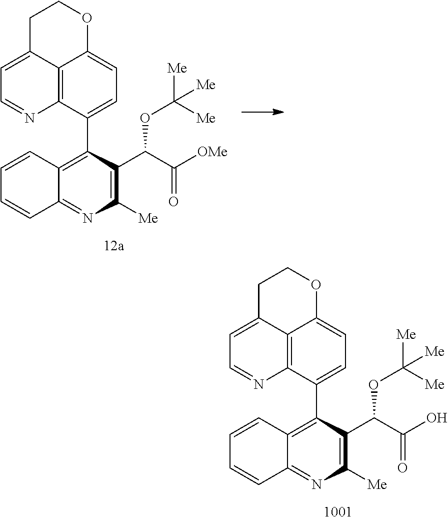

To a suitable clean and dry reactor under a nitrogen atmosphere was charged 12a (9.69 Kg, 21.2 mol) and ethanol (23.0 Kg). The mixture was agitated and the batch temperature was maintained at Tᵢₙₜ=20 to 25° C. 2 M sodium hydroxide (17.2 Kg) was charged at Tᵢₙₜ=20 to 25° C. and the batch temperature was adjusted to Tᵢₙₜ=60-65° C. over no less than 30 minutes. The batch was agitated at Tᵢₙₜ=60-65° C. for 2-3 hours until HPLC conversion was >99.5% area (12a is <0.5 area %). The batch temperature was adjuted to Tᵢₙₜ=50 to 55° C. and 2M aqueous HCl (14.54 Kg) was charged. The pH of the batch was adjusted to pH 5.0 to 5.5 (target pH 5.2 to 5.3) via the slow charge of 2M aqueous HCl (0.46 Kg) at Tᵢₙₜ=50 to 55=C. Acetonitrile was charged to the batch (4.46 Kg) at Tᵢₙₜ=50 to 55° C. A slurry of seed crystals (1001, 20 g in 155 g of acetonitrile) was charged to the batch at Tᵢₙₜ=50 to 55° C. The batch was agitated at Tᵢₙₜ=50 to 55° C. for no less than 1 hour (1-2 hours). The contents were vacuum distilled to ˜3.4 vol (32 L) while maintaining the internal temperature at 45-55° C. A sample of the batch was removed and the ethanol content was determined by GC analysis; the criterion was no more than 10 wt % ethanol. If the ethanol wt % was over 10%, an additional 10% of the original volume was distilled and sampled for ethanol wt %. The batch temperature was adjusted to Tᵢₙₜ=18-22° C. over no less than 1 hour. The pH of the batch was verified to be pH=5-5.5 and the pH was adjusted, if necessary, with the slow addition of 2 M HCl or 2 M NaOH aqueous solutions. The batch was agitated at Tᵢₙₜ=18-22° C. for no less than 6 hours and the solids were collected by filtration. The filtrate/mother liquid was used to remove all solids from reactor. The cake with was washed with water (19.4 Kg) (water temperature was no more than 20° C.). The cake was dried under vacuum at no more than 60° C. for 12 hours or until the LOD was no more than 4% to obtain 1001 (9.52 Kg, 99.6 A % 220 nm, 97.6 wt % as determined by HPLC wt % assay, 99.0% yield).

### Example 14

Hydrochloride Salt of Compound (I), Type A

Compound (I) (263 mg) was added to a vial of ethanol (1.5 mL), and then 36.5% HCL aqueous solution (59 mg) was added. The mixture was heated to 70° C.; and stirred at this temperature until solid material was obtained. The mixture was cooled to 20° C. over a period of 10 hours. After cooling, isopropanol (400 μL) was added over a period of 3 hours. The resulting solids were collected and characterized as the hydrochloride salt of Compound (I), Type A.

The hydrochloride salt of Compound (I), Type A was prepared analogously to the aforementioned procedure using methyl ethyl ketone, tetrahydrofuran, acetonitrile, ethyl acetate, dichloroethane and methyl-t-buyl ether instead of ethanol.

### Example 15

Hydrochloride Salt of Compound (I), Type B

Compound (I) (40 mg) was added to a vial of tetrahydrofuran (500 μL) and water (100 μL). 36.5% HCL aqueous solution (˜10 mg) was added to the mixture. The vial is evaporated to dryness, and toluene (1 mL) was added. The mixture was stirred overnight. The resulting solids were collected and characterized as the hydrochloride salt of Compound (I), Type B with toluene.

The hydrochloride salt of Compound (I), Type B may also be prepared analogously to the aforementioned procedure using anisole instead of toluene.

### Example 16

Compound (I), Form I

Compound (I) (15.36 mg) was added to 150 μL acetonitrile at room temperature. The mixture was stirred overnight. The mixture was heated to 70° C. at a rate of 2° C./minute and was held at this temperature for 30 minutes. The mixture was cooled to 20° C. at a rate of 0.2° C./minute. The mixture was stirred at room temperature for about 96 hours. The resulting solids were collected and characterized as Compound (I), Form I with acetonitrile.

Compound (I), Form I may also be prepared analogously to the aforementioned procedure using acetone, methanol, ethanol instead of acetonitrile.

### Example 17

Compound (I), Form II

Compound (I) (150 mg) was added to 1.5 mL methyl-t-butyl ether (with 1.5% water) at room temperature. The mixture was heated to 50° C. to provide a solution. The solution was cooled to 20° C. and stirred over a period of 4 hours. The solution was stirred for an additional 48 hours at 20° C. and crystals precipitated while stirring. The resulting solids were collected and characterized as Compound (I), Form II with methyl-t-butyl ether.

Compound (I), Form II may also be prepared analogously to the aforementioned procedure using butyl acetate instead of methyl-t-butyl ether

### Example 18

Compound (I), Form III

Compound (I), Form II (250 mg) was added to water (15 mL). The mixture was heated to 80° C. to provide a slurry which was then stirred at 80° C. for 8 hours. After being cooled to 20° C. over 2 hours, the solids were collected and characterized as Compound (I), Form III.

### Example 19

Hydrochloride Salt of Compound (I), Type A

In a suitable reactor, Compound (I) (30 g, 95.6 wt %) was dissolved in 135 mL of ethanol (200 proof, SDA2B grade, denatured with toluene) at approximately 78° C. The solution was polish filtered and distilled at reduced pressure (approximately 60-65° C. and 200-250 Torr) to a volume of approximately 75-95 mL. The solution temperature was then adjusted to 50±2° C. for the partial addition of a dilute solution of hydrochloric acid in isopropyl alcohol (IPA). Approximately 1.05 equivalents of anhydrous HCl (75 mL, 0.905M in IPA) was prepared for the addition. After about 30-40% of the dilute HCl solution (22-30 mL) was charged, the solution was seeded with Type A crystals of the hydrochloride salt of Compound (I) (approximately 0.15 g, 0.5 wt %). Crystallization slowly proceeded upon the seed addition, and after aging the batch at 50±5° C. for at least 0.5 hr, a crystal slurry bed was formed. The remaining 60-70% of the HCl solution (45-52 mL) was slowly charged to the batch over at least 1-5 hr at 50±5° C. The product was further crystallized out of solution with the addition of heptane (150 mL, 103 g) slowly over at least 1-5 hr at 50±5° C. The batch was then cooled to 10±5° C. linearly over at least 2-5 hr and the slurry was aged at 10±5° C. for at least 2 hr. The slurry was filtered and the cake washed with 100 mL of SDA2B EtOH/Heptane mixture (1:5 v/v or 13.2 g:57.0 g). The cake was dried at 60±5° C. and ≦100 mm Hg for at least 24 hr (until EtOH, IPA and heptane ≦0.5% (GC analysis) to provide 29.69 g of the hydrochloride salt of Compound (I), Type A (95% yield, purity=99.76 area % by HPLC and 99.86 ee).

### Example 20

Preparation of 12b

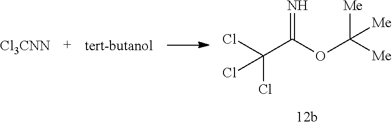 [SMILES: "Cl3CNN" ; "+" ; "tert-butanol" ; CC(C)(C)OC(=N)C(Cl)(Cl)Cl]

To a 2 L 3-neck dried reactor under a nitrogen atmosphere was charged 3 mol % (10.2 g, 103 mmol) of sodium tert-butoxide and 1.0 equivalent of tert-butanol (330.5 mL, 3.42 mol). The batch was heated at Tᵢₙₜ=50 to 60° C. until most of the solid was dissolved (˜1 to 2 h). Fluorobenzene (300 mL) was charged to the batch. The batch was cooled to Tᵢₙₜ=<−5° C. (−10 to −5° C.) and 1.0 equivalent of trichloroacetonitrile (350 mL, 3.42 mol) was charged to the batch. The addition was exothermic so the addition was controlled to maintain Tᵢₙₜ=<−5° C. The batch temperature was increased to Tᵢₙₜ=15 to 20° C. and heptane (700 mL) was charged. The batch was agitated at Tᵢₙₜ=15 to 20° C. for no less than 1 h. The batch was passed through a short Celite (Celite 545) plug to produce 1.256 Kg of 12b. Proton NMR with the internal standard indicated 54.6 wt % 12b, 27.8 wt % heptane and 16.1 wt % fluorobenzene (overall yield: 92%).

Each reference, including all patents, patent applications, and publications cited in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application.

1. A hydrochloride salt of Compound (I): 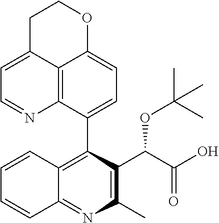
in crystalline form Type A.

2. (canceled)

3. The crystalline hydrochloride salt of Compound (I) according to claim 1 in crystalline Type A having an X-ray powder diffraction pattern comprising peaks at 8.1, 9.3, 11.2, 28.4 and 28.6 degrees 2θ (±0.2 degrees 2θ) when measured using CuKα radiation.

4. The crystalline hydrochloride salt of Compound (I) according to claim 3 in crystalline Type A having an X-ray powder diffraction pattern further comprising a peak at 13.0 degrees 2θ (±0.2 degrees 2θ) when measured using CuKα radiation.

5. The crystalline hydrochloride salt of Compound (I) according to claim 4 in crystalline Type A having an X-ray powder diffraction pattern further comprising peaks at 10.4, 12.1, 18.8, 19.8, 22.1 and 22.4 degrees 2θ (±0.2 degrees 2θ) when measured using CuKα radiation.

6. The crystalline hydrochloride salt of Compound (I) according to claim 1 in crystalline Type A having an X-ray powder diffraction pattern substantially the same as that shown in FIG. 1.

7. The crystalline hydrochloride salt of Compound (I) according to claim 1 in crystalline Type A having a DSC thermal curve substantially the same as that shown in FIG. 2 indicated as Type A.

8. The crystalline hydrochloride salt of Compound (I) according to claim 1 in crystalline Type A having an X-ray powder diffraction pattern comprising peaks at 8.1, 9.3, 11.2, 28.4 and 28.6 degrees 2θ (±0.2 degrees 2θ) when measured using CuKα radiation and having a DSC thermal curve substantially the same as that shown in FIG. 2 indicated as Type A.

9. The crystalline hydrochloride salt of Compound (I) in crystalline Type A according to claim 1 having a ¹³C-ssNMR spectrum having chemical shift peaks at 146.7, 140.4, 136.9, 123.1, 121.4, and 21.8 ppm (each peak is ±0.2 ppm).

10. The crystalline hydrochloride salt of Compound (I) according to claim 9 in crystalline Type A having a ¹³C-ssNMR spectrum further comprising a chemical shift peak at 171.0 ppm (±0.2 ppm).

11. The crystalline hydrochloride salt of Compound (I) according to claim 10 in crystalline Type A having a ¹³C-ssNMR spectrum further comprising chemical shift peaks at 158.7, 154.2, 150.5 and 28.7 ppm (each peak is ±0.2 ppm).

12. The crystalline hydrochloride salt of Compound (I) according to claim 11 in crystalline Type A having a ¹³C-ssNMR spectrum further comprising chemical shift peaks at 133.0, 129.8, 128.8, 125.8, 118.5, 115.9, 110.7, 78.1, 72.2 and 65.2 ppm (each peak is ±0.2 ppm).

13. The crystalline hydrochloride salt of Compound (I) according to claim 1 in crystalline Type A having an X-ray power diffraction pattern comprising peaks at 8.1, 9.3, 11.2, 28.4 and 28.6 degrees 2θ (±0.2 degrees 2θ) when measured using CuKα radiation and a ¹³C-ssNMR spectrum having chemical shift peaks at 146.7, 140.4, 136.9, 123.1, 121.4, and 21.8 ppm (each peak is ±0.2 ppm).

14. The crystalline hydrochloride salt of Compound (I) according to claim 1 in crystalline Type A having a ¹³C-ssNMR spectrum substantially the same as that shown in FIG. 3.

15-48. (canceled)

49. A pharmaceutical composition comprising a hydrochloride salt of Compound (I) according to claim 1, and at least one pharmaceutically acceptable carrier or diluent.

50. (canceled)

51. The pharmaceutical composition according to claim 49 further comprising at least one other antiviral agent.

52. (canceled)

53. A process to prepare crystalline form Type A of the hydrochloride salt of Compound (I) according to claim 1 comprising the following steps:
(i) dissolving Compound (I) in a suitable solvent, and then adding an aqueous solution of HCl;
(ii) slowly heating the mixture in step (i) with stirring to a temperature to obtain a solution or slurry;
(iii) slowly cooling the mixture obtained in step (ii);
(iv) slowly adding an anti-solvent; and
(v) collecting the solid material obtained in step (iv) to obtain the hydrochloride salt of Compound (I) according to claim 1.

54. A process to prepare crystalline form Type A of the hydrochloride salt of Compound (I) according to claim 1 comprising the following steps:
(a) dissolving Compound (I) in a suitable solvent at a temperature greater than room temperature and then polish-filtering;
(b) optionally, adjusting the solution volume;
(c) cooling the solution temperature;
(d) adding dilute HCl in water or an aliphatic alcohol;
(e) initiating crystallization by seeding with crystals of the hydrochloride salt of Compound (I) according to claim 1;
(f) continuing crystallization by controlled slow addition of dilute HCl in water or an aliphatic alcohol;
(g) crystallizing the product further out of solution with the addition of non-polar solvents; and
(h) filtering and drying to provide crystals of the hydrochloride salt of Compound (I) according to claim 1.

55-58. (canceled)

59. A method of treating or preventing an HIV infection in a human having or at risk of having the infection by administering to the human a therapeutically effective amount of a hydrochloride salt of Compound (I) according to claim 1 or a pharmaceutical composition according to claim 49.
